(19) United States
(12) Patent Application Publication
(10) Pub. No.: US 2013/0305396 A1
(43) Pub. Date: Nov. 14, 2013

(54) ENGINEERED HUMAN ENDOSIALIN-EXPRESSING RODENTS

(71) Applicants: Luigi Grasso, Bryn Mawr, PA (US); Jian Min Lin, Malvern, PA (US); Yuhong Zhou, Phoenixville, PA (US); Brian E. Tomkowicz, East Norriton, PA (US); Nicholas C. Nicolaides, Glen Mills, PA (US); Philip M. Sass, Audubon, PA (US)

(72) Inventors: Luigi Grasso, Bryn Mawr, PA (US); Jian Min Lin, Malvern, PA (US); Yuhong Zhou, Phoenixville, PA (US); Brian E. Tomkowicz, East Norriton, PA (US); Nicholas C. Nicolaides, Glen Mills, PA (US); Philip M. Sass, Audubon, PA (US)

(21) Appl. No.: 13/876,232

(22) PCT Filed: Sep. 28, 2011
(86) PCT No.: PCT/US11/53651

§ 371 Date: Jun. 11, 2013

Related U.S. Application Data
(60) Provisional application No. 61/387,898, filed on Sep. 29, 2010.

(51) Int. Cl. A61K 49/00

(52) U.S. Cl. CPC A61K 49/0008
USPC 800/3; 800/18; 435/354; 435/7.23

(57) ABSTRACT

Provided herein are rodents that express the human endosialin gene. In preferred embodiments, the rodent is a mouse. Preferably, the human endosialin gene is integrated into the native or endogenous endosialin gene locus. More preferably, the host rodent is null for the endogenous endosialin gene product. The human endosialin gene is preferably expressed in a similar development and disease response pattern as that of the native endosialin gene product in parental or wild type rodents. This feature makes these rodents useful for studying the effects of test agents to positively or negatively affect endosialin biology for therapeutic use. Use of human endosialin expressing rodents lacking native endosialin gene product (HUE rodents) is proposed as a strategy for developing agents that can positively or negatively affect the endosialin pathway and also serve as a screening tool to identify those agents that may be useful as human therapies.

### CROSS-REFERENCE TO RELATED APPLICATIONS

This application claims the benefit of U.S. Application No. 61/387,898, filed Sep. 29, 2010, which application is incorporated by reference herein in its entirety.

### TECHNICAL FIELD

Provided herein is technology relating to transgenic rodents containing human endosialin and their development and use. The rodents and derived organs, cells and biological fluids are useful in the elucidation of endosialin pathways, for example in development and in normal and disease states, as well as the development of endosialin specific compounds that may block or stimulate endosialin expression/function for therapeutic benefit. The rodents may be used to determine pathways and gene elements that can regulate endosialin function during development, homeostasis, or disease for developing agents that can positively or negatively regulate human endosialin function(s).

### BACKGROUND

Angiogenesis is a regulated process involving the formation of new blood vessels. It plays an essential role in normal growth, embryonic development, wound healing, and other physiological processes (Yancopoulos et. al. (2000) Nature 407(6801):242-8). Moreover, de novo angiogenesis is involved in several disease states including cancer, where the formation of new “embryonic-like” blood vessels (referred to as neovascularization herein) appear that differ from normal vasculature with regards to structure and function (Hanahan and Weinberg, (2000) Cell 100(1):57-70; Peters et. al. (2005) Nat. Med. 11:261-262). These vessels have been found to include cells such as endothelial cells, pericytes and fibroblasts. A number of in vivo and in vitro studies have demonstrated biological differences between normal and disease-associated vasculature as determined using various model systems of angiogenesis offering the ability to develop novel anti-angiogenic compounds that can selectively inhibit vessel formation of the embryonic-type, tumor-associated endothelia for therapy of neovascular disease (Dhanabal et. al. (2005) AntiCancer Agents 5:115-130). Neovascular diseases include cancer, opthalmologic indications, inflammation and infectious disease (Conejo-Garcia, et. al. (2005) Blood 105:679-681; Das et. al. (2003) Prog. Retin. Eye Res. 22:721-748; Paleolog et. al. (1999) Angiogenesis 2:295-307; Wilkinson-Berka, et. al. (2004) Cur. Pharm. Des. 10:3331-3348). In addition, diseases exist whereby enhanced angiogenesis may improve the outcome of the disease (Galiano, et. al. (2004) Am. J. Path. 164:1935-1947; Wang et. al. (2004) Stroke 35:1732-1737). In light of these opportunities for therapy, an intense search for potential targets that can specifically inhibit or stimulate disease-associated neovascularization, vessel growth and/or suppression, and function is ongoing. In an attempt to identify such targets, strategies have been designed to identify cell surface antigens of tumor stroma as well as isolate specific proteins or RNA that are expressed in neovascular endothelial and endothelial associated cells (Rettig et. al. (1992) Proc Natl. Acad Sci USA 89(22):10832-6; St. Croix, et. al. (2000) Science 289: 1197-1202). These strategies have identified a cell surface protein that appears to be specifically expressed in pericytes and other tumor stromal cells, called endosialin, CD248 or tumor endothelial marker-1 (TEM1). Studies employing antibodies that can bind to endosialin have identified a subset of cells that express this antigen in endothelial cell cultures as well as a subset of cells in normal tissue of patients. Immunohistochemistry (IHC) studies of malignant tissues have revealed good expression of endosialin in a number of neovascular associated cells in malignant tissues. Expression of endosialin in cell lines derived from embryonic-like endothelial cultures, such as but not limited to HUVEC (Human Umbilical Vein Endothelial Cells) or HMVEC-(Neonatal Dermal Microvascular Endothelial Cells) and primary human pericytes, has also been observed. Endosialin-expressing cells appear to be fibroblastic-like in morphology. Recent studies have found that endosialin is at least expressed in pericytes associated with tumor vasculature (Tomkowicz et al. (2010) Cancer Biol. Ther. 9:1-8).

In 1992, Rettig et al. described monoclonal antibodies that recognize antigens on vessels within various cancer types (Rettig et. al. (1992) Proc Natl. Acad Sci USA 89(22):10832-6). One of these was designated FB5, which recognizes a ˜100 kDa protein on the surface of a neuroblastoma cell line, LA1-5s. FB5 is a murine, IgG1, antibody that binds to endosialin and has been shown to recognize cells associated with tumor vasculature and stromal cells associated with a variety of different cancer types. Structural evaluation classified endosialin as a C-type lectin-like protein, composed of five globular extracellular domains (including a C-type lectin domain, one domain with similarity to the Sushi/ccp/scr pattern, and three EGF repeats). The protein also contains a mucin-like region, a transmembrane segment, and a short cytoplasmic tail. The protein appears to be a glycoprotein. Carbohydrate analysis shows that the endosialin core protein has an abundance of sialylated, O-linked oligosaccharides, with similarities to sialomucin-like molecules.

U.S. Pat. No. 5,342,757 describes an antibody that binds to a ˜100 kDa protein (endosialin). The antibody was named FB5. Subsequent work combined the complementarity determining regions (CDR) of the mouse FB5 onto a human IgG1 backbone to create a humanized antibody that is able to also bind to vessels within malignant tissues and a subset of cells in HMVEC cultures.

U.S. Pat. No. 7,615,372 describes an antibody that binds to endosialin and can elicit immune effector activity and internalize.

Neovascularization is associated with a number of disease states. In cancer it is believed that neovascularization is important to supply tumors with blood and nutrients. In non-oncology diseases such as retinopathy and macular degeneration, uncontrolled neovascularization causes loss of sight (Wilkinson-Berka, (2004) Curr Pharm Des. 10(27):3331-48; Das and McGuire, (2003) Prog Retin Eye Res. 22(6):721-48). Moreover, several reports have identified a role of neovascularization in inflammatory disease (Paleolog and Miotla, (1998) Angiogenesis 2(4):295-307). Methods to better define the embryonic-like endothelial and precursor cells as well as methods to study cells that associate with these endothelial cells involved with the aforementioned disease states will lead to the development of novel drugs to treat these diseases. Conversely, neovascularization is associated with wound healing (Galiano et. al. (2004) Am J. Pathol. 164(6):1935-47). Many of these complex processes are difficult to study in vitro, therefore an in vivo model that can test the effects of agents that can affect the human endosialin protein will enable the development, refinement and validation for understanding the role of this pathway in humans and potentially lead to the identification of agents that can be used for therapeutic benefit including compounds to treat cancer, inflammatory disease as well as those that can enhance wound treatment associated with trauma, burns and infection.

### SUMMARY

In vivo models are valuable for studying complex, multicellular processes. The degree of homology between human and endosialin gene products of other species varies, with rodent species sharing less than 80% homology (Opavsky et al., J Biol. Chem. 2001 Oct. 19; 276(42):38795-807; Carson-Walter et al., Cancer Res. 2001 Sep. 15; 61(18):6649-55). Therefore animal models that express the human endosialin gene product would be beneficial for identifying and developing agents that can bind to human endosialin mRNA or protein to study its biological roles in angiogenesis and neovascular disease as well as other biological processes such as inflammation, ophthalmologic disease, wound healing and animal development. The use of animal models expressing human endosialin has not been described previously. Moreover the development of human endosialin expressing rodents whereby the human gene is substituted for the rodent homolog within the natural rodent genome and where the human gene expression behaves identically as the endogenous rodent offers an important use for these animals in the study of human endosialin biology in normal tissues as well as in various disease states and in the development of agents that can bind to the human gene product in vivo.

Provided herein are rodents that express a nucleotide sequence encoding human endosialin (e.g., SEQ ID NOs: 3 and 4). In preferred embodiments, the rodent is a mouse. Murine endosialin nucleotide and amino acid sequences are provided in SEQ ID NOs: 1 and 2, respectively. Preferably, the nucleotide sequence encoding human endosialin is integrated into the native or endogenous endosialin gene locus. More preferably, the endogenous endosialin gene product is functionally disrupted or is substantially reduced or null in the transgenic rodent. Preferably, the transgenic rodent comprises a nucleotide sequence encoding human endosialin that is under the control of the rodent's endogenous gene expression regulatory sequences. In some embodiments, the construct containing the human endosialin-encoding nucleotide sequence also contains a reporter gene or a selectable marker (e.g., a positive selection marker and/or a negative selection marker). The nucleotide sequence encoding human endosialin is preferably expressed in a similar development and disease response pattern as that of the native endosialin gene product in parental or wild type rodents. This feature makes these rodents useful for studying the effects of test agents to positively or negatively affect endosialin biology for therapeutic use. Use of human endosialin expressing rodents lacking native endosialin gene product (HUE rodents) is proposed as a strategy for developing agents that can positively or negatively affect the endosialin pathway and also serve as a screening tool to identify those agents that may be useful as human therapies.

Rodents expressing a nucleotide sequence encoding human endosialin from the endogenous endosialin locus whereby the human endosialin sequence is expressed similarly as the rodent endosialin sequence during various physiological processes are provided.

Provided are the progeny of the transgenic rodents expressing human endosialin.

Provided are cells isolated from the transgenic rodents expressing human endosialin. These cells can be isolated from normal tissue, malignant tissue, inflamed tissue or diseased eye.

Also provided are methods of using the human endosialin expressing rodents, referred to as HUE rodents, to study the biology of the human endosialin and to develop agents that can affect endosialin biology as it relates to primary endothelial cell and pericyte populations, malignant tissues or normal tissues as well as other diseases.

In some embodiments, the mice of the invention have a nucleotide sequence encoding human endosialin knocked into the mouse endosialin locus on the proximal region of mouse chromosome 19.

In some embodiments, the mice consist of a C57BL/6 strain, whereby studies of human endosialin can be conducted in these mice or mice in which the human endosialin locus is crossed to other mouse strains as commonly done by methods known to those skilled in the art.

Provided is a method of studying the role of human endosialin in normal development comprising measuring the level of expression of the human endosialin in the organs, tissues or cells of a transgenic rodent comprising a nucleotide sequence encoding human endosialin integrated into the genome of the rodent. In preferred embodiments, the level of expression of the human endosialin is measured at a particular developmental stage of the rodent.

Also provided is a method of studying the role of human endosialin function in normal development comprising localizing the expression of the human endosialin in the organs, tissues or cells of a transgenic rodent comprising a nucleotide sequence encoding human endosialin integrated into the genome of the rodent. In preferred embodiments, the nucleotide sequence comprises a reporter gene or selectable marker.

Provided is a method of studying the role of human endosialin in supporting or preventing a disease phenotype comprising measuring the level of expression of the human endosialin in cells of a transgenic rodent comprising a nucleotide sequence encoding human endosialin integrated into the genome of the rodent wherein the rodent exhibits the disease phenotype. In preferred embodiments the phenotype is cancer. In some preferred embodiments the phenotype is inflammatory disease. In further preferred embodiments the phenotype is eye disease. In yet further preferred embodiments the phenotype is reduced wound healing.

Provided is a method of studying the role of human endosialin in supporting or preventing a dysplastic phenotype comprising grafting a tumor onto a transgenic rodent comprising a nucleotide sequence encoding human endosialin integrated into the genome of the rodent and measuring the level of expression of the human endosialin in cells of the rodent.

Also provided is a method of studying the role of human endosialin in supporting or preventing a dysplastic phenotype comprising grafting a tumor onto a transgenic rodent comprising a nucleotide sequence encoding human endosialin integrated into the genome of the rodent and localizing the expression of the human endosialin in cells of the rodent. In preferred embodiments, the nucleotide sequence comprises a reporter gene or selectable marker.

Provided is a method of studying the role of human endosialin in supporting or preventing a dysplastic phenotype comprising grafting a tumor onto the progeny of a transgenic rodent comprising a nucleotide sequence encoding human endosialin integrated into the genome of the rodent and an immunocompromised rodent and measuring the level of expression of the human endosialin in cells of the progeny. In preferred embodiments, the nucleotide sequence comprises a reporter gene or selectable marker.

Also provided is a method of screening test pharmacological agents to identify a targeting agent for human endosialin comprising administering a test pharmacological agent to a transgenic rodent comprising a nucleotide sequence encoding human endosialin integrated into the genome of the rodent, measuring human endosialin activity in the rodent and comparing the human endosialin activity to a control. An increase or decrease in human endosialin activity relative to the control can be indicative of a targeting agent for endosialin. Also provided is a method of validating an agent for human endosialin comprising administering the agent to a transgenic rodent comprising a nucleotide sequence encoding human endosialin integrated into the genome of the rodent, measuring human endosialin activity in the rodent and comparing the human endosialin activity to a control. An increase or decrease in human endosialin activity relative to the control can validate the agent for endosialin. In preferred embodiments of the methods described herein, the targeting agent is an antibody or antigen-binding fragment thereof. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.). In some preferred embodiments, the agent can stimulate human endosialin activity. In further preferred embodiments, the agent can suppress human endosialin activity. In yet further preferred embodiments, the targeting agent is an endosialin binding protein. In some embodiments, the binding protein can stimulate human endosialin activity. In some embodiments, the binding protein can suppress human endosialin activity. In some embodiments, the binding protein is bound to an agent that stimulates or suppresses human endosialin activity. In some preferred embodiments, the agent is a nucleic acid that can complementary bind to human endosialin mRNA. In some preferred embodiments, the agent is a small chemical molecule. In some embodiments, the test agent is bound to a detectable label. Exemplary detectable labels include but are not limited to chemiluminescent compounds (e.g., an acridinium ester compound), a phosphorescent compound, a fluorescent compound, a radiolabel, biotin, or an enzyme.

Further provided is a method of screening test pharmacological agents to identify a targeting agent for human endosialin comprising contacting a test pharmacological agent to a cell of a transgenic rodent comprising a nucleotide sequence encoding human endosialin integrated into the genome of the rodent measuring human endosialin activity in the cell and comparing the human endosialin activity to a control. An increase or decrease in human endosialin activity relative to the control can be indicative of a targeting agent for endosialin. Also provided is a method of validating an agent for human endosialin comprising contacting said agent to a cell of a transgenic rodent comprising a nucleotide sequence encoding human endosialin integrated into the genome of said rodent, measuring human endosialin activity in the cell and comparing the human endosialin activity to a control. An increase or decrease in human endosialin activity relative to the control can validate the agent for endosialin. In preferred embodiments of the methods described herein, the targeting agent is an antibody or antigen-binding fragment thereof. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.). In some preferred embodiments, the agent can stimulate human endosialin activity. In further preferred embodiments, the agent can suppress human endosialin activity. In yet further preferred embodiments, the targeting agent is an endosialin binding protein. In some embodiments, the binding protein can stimulate human endosialin activity. In some embodiments, the binding protein can suppress human endosialin activity. In some preferred embodiments, the agent is a nucleic acid that can complementary bind to human endosialin mRNA. In some preferred embodiments, the agent is a small chemical molecule. In some embodiments, the test agent is bound to a detectable label. Exemplary detectable labels include but are not limited to chemiluminescent compounds (e.g., an acridinium ester compound), a phosphorescent compound, a fluorescent compound, a radiolabel, biotin, or an enzyme.

Provided is a method of screening test pharmacological agents to identify a targeting agent for human endosialin comprising administering a test pharmacological agent to a transgenic rodent comprising a nucleotide sequence encoding human endosialin integrated into the genome of the rodent, measuring human PDGF receptor pathway activity in endosialin expressing cells of the rodent and comparing the human PDGF receptor pathway activity to a control. In some preferred embodiments, the agent stimulates PDGF receptor pathway in endosialin expressing cells. In some preferred embodiments, the agent suppresses PDGF receptor pathway in endosialin expressing cells. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method of screening test pharmacological agents to identify a targeting agent for human endosialin comprising contacting a test pharmacological agent to a cell of a transgenic rodent comprising a nucleotide sequence encoding human endosialin integrated into the genome of the rodent, measuring human PDGF receptor pathway activity in the cell and comparing the human PDGF receptor pathway activity to a control. In some preferred embodiments, the agent stimulates PDGF receptor pathway in endosialin expressing cells. In some preferred embodiments, the agent suppresses PDGF receptor pathway in endosialin expressing cells. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for screening for test agents that can suppress disease comprising administering a test agent to a transgenic rodent comprising a nucleotide sequence encoding human endosialin integrated into the genome of the rodent wherein the rodent exhibits the disease, measuring presence of the disease in the transgenic rodent and comparing presence of the disease in the transgenic rodent to a control. A decrease in the disease relative to the control can be indicative of an agent that can suppress the disease. Also provided is a method for validating an agent that can suppress disease comprising administering the agent to a transgenic rodent comprising a nucleotide sequence encoding human endosialin integrated into the genome of the rodent wherein the rodent exhibits the disease, measuring presence of the disease in the transgenic rodent, and comparing presence of the disease in the transgenic rodent to a control. A decrease in the disease relative to the control can validate an agent that can suppress the disease. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.). In preferred embodiments, the disease is cancer. In other preferred embodiments the disease is inflammatory disease. In further preferred embodiments the disease is eye disease. In yet further preferred embodiments the disease is reduced wound healing.

Further provided is a method for screening for test agents that can suppress disease comprising contacting a test agent to a cell of a transgenic rodent comprising a nucleotide sequence encoding human endosialin integrated into the genome of the rodent wherein the rodent exhibits the disease, measuring presence of the disease in the cell and comparing presence of the disease in the cell to a control. A decrease in the disease relative to the control can be indicative of an agent that can suppress the disease. Also provided is a method for validating an agent that can suppress disease comprising contacting the agent to a cell of a transgenic rodent comprising a nucleotide sequence encoding human endosialin integrated into the genome of the rodent wherein the cell exhibits the disease, measuring presence of the disease in the cell, and comparing presence of the disease in the cell to a control. A decrease in the disease relative to the control can validate an agent that can suppress the disease. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.). In preferred embodiments, the disease is cancer. In other preferred embodiments the disease is inflammatory disease. In further preferred embodiments the disease is eye disease. In yet further preferred embodiments the disease is reduced wound healing. Also provided is optionally a tumor can be grafted onto the transgenic rodent. Also provided is optionally the transgenic rodent has metastases.

Provided is a method for screening for test agents that can suppress tumor growth comprising grafting a tumor onto a transgenic rodent comprising a nucleotide sequence encoding human endosialin integrated into the genome of the rodent, administering a test agent to the transgenic rodent, measuring tumor growth in the transgenic rodent and comparing tumor growth in the transgenic rodent to a control. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method for screening for test agents that can suppress tumor growth comprising grafting a tumor onto the progeny of a transgenic rodent comprising a nucleotide sequence encoding human endosialin integrated into the genome of the rodent and an immunocompromised rodent, administering a test agent to the transgenic rodent, measuring tumor growth in the transgenic rodent and comparing tumor growth in the transgenic rodent to a control. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for screening for test agents that can suppress tumor growth comprising grafting a tumor onto a transgenic rodent comprising a nucleotide sequence encoding human endosialin integrated into the genome of the rodent, administering a test agent to the transgenic rodent, localizing tumor cells in the transgenic rodent and comparing the localization of tumor cells in the transgenic rodent to a control. In preferred embodiments, the nucleotide sequence comprises a reporter gene. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method for screening for test agents that can suppress tumor growth comprising grafting a tumor onto the progeny of a transgenic rodent comprising a nucleotide sequence encoding human endosialin integrated into the genome of the rodent and an immunocompromised rodent, administering a test agent to the transgenic progeny rodent, localizing tumor cells in the transgenic progeny rodent and comparing tumor cell localization in the transgenic progeny rodent to a control. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for determining the optimal dosage of an agent, for example, an agent identified by either of the previous screening methods, in treating the disease comprising administering the agent to the transgenic rodent at different dosages, measuring the presence of the disease in the transgenic rodent and determining at which dosage the agent suppresses the disease in the transgenic rodent. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method for determining the optimal dosage of an anti-endosialin antibody or antigen-binding fragment thereof in treating a disease comprising administering the antibody or antigen-binding fragment thereof at different dosages to a transgenic rodent comprising a nucleotide sequence encoding human endosialin integrated into the genome of the rodent, measuring the presence of the disease in the transgenic rodent and determining at which dosage the agent suppresses the disease in the transgenic rodent. In preferred embodiments, the disease is cancer, inflammatory disease, eye disease or reduced wound healing.

Provided is a vector comprising the human endosialin open reading frame cloned in between genomic regions endogenous to the rodent endosialin locus.

Also provided is a cell isolated from a transgenic rodent comprising a nucleotide sequence encoding human endosialin integrated into the genome of the rodent. In preferred embodiments the cell is isolated from normal tissue. In other preferred embodiments the cell is isolated from malignant tissue. In further preferred embodiments the cell is isolated from inflamed tissue. In yet further preferred embodiments the cell is isolated from diseased eye.

Provided is a method for screening for gene expression with a cell isolated from a transgenic rodent comprising a nucleotide sequence encoding human endosialin integrated into the genome of the rodent, comprising measuring an expression profile in the cell. In some embodiments, gene expression is measured before, simultaneously with, and/or after administration of a test agent. In preferred embodiments the expression profile is measured directly after isolation of the cell. In some preferred embodiments the expression profile is measured after culturing the cell in the presence of stimulants or nutrients. In further preferred embodiments the expression profile is measured after treatment with a test agent. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.). In some preferred embodiments the cell is analyzed for RNA profiles. In further preferred embodiments the cell is analyzed for cDNA profiles. In yet further preferred embodiments the cell is analyzed for protein profiles.

Provided is a method for screening test agents that inhibit the growth and/or differentiation of endosialin expressing cells comprising administering a test agent to the cell isolated from a transgenic rodent comprising a nucleotide sequence encoding human endosialin integrated into the genome of the rodent, measuring the growth and/or differentiation of the cell and comparing the growth and/or differentiation to a control untreated cell, wherein the test agent inhibits the growth and/or differentiation of the cell if the level of growth and/or differentiation of the cell is lower than the level of growth and/or differentiation of the control untreated cell. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for screening for test agents that stimulate the growth and/or differentiation of endosialin expressing cells comprising administering a test agent to the cell isolated from a transgenic rodent comprising a nucleotide sequence encoding human endosialin integrated into the genome of the rodent, measuring the growth and/or differentiation of the cell and comparing the growth and/or differentiation to a control untreated cell, wherein the test agent stimulates the growth and/or differentiation of the cell if the level of growth and/or differentiation of the cell is higher than the level of growth and/or differentiation of the control untreated cell. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method for screening for test agents that inhibit normal biological activity of endosialin expressing cells comprising administering a test agent to the cell isolated from a transgenic rodent comprising a nucleotide sequence encoding human endosialin integrated into the genome of the rodent, measuring the biological activity of the cell and comparing the biological activity to a control untreated cell, wherein the test agent inhibits the biological activity of the cell if the level of biological activity of the cell is lower than the level of biological of the control untreated cell. In some embodiments the biological activity of the cell comprises the binding of endosialin to collagen Col I, Col IV or fibronectin. In some embodiments the biological activity of the cell comprises cell adhesion to extracellular matrices. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

In preferred embodiments of the previous methods, the agent is an antibody. In further preferred embodiments the antibody is a monoclonal antibody. In some preferred embodiments the antibody is a polyclonal antibody. In some preferred embodiments the antibody is an antigen-binding fragment of an antibody. In further preferred embodiments the antibody contains 1-6 CDRs that can bind to human endosialin.

In preferred embodiments of the previous methods, the agent is a protein. In further preferred embodiments the protein is a full length protein. In some preferred embodiments the protein is a peptide. In some preferred embodiments the protein is a mimetic.

In preferred embodiments of the previous methods, the agent is a chemical. In further preferred embodiments the chemical stimulates endosialin gene expression. In some preferred embodiments the chemical suppresses endosialin gene expression. In some preferred embodiments the chemical suppresses endosialin protein function. In further preferred embodiments the chemical stimulates endosialin protein function.

In preferred embodiments of the previous three methods, the agent is an antisense oligonucleotide. In further preferred embodiments the agent is an RNAi molecule.

Provided is a method for testing agents for detecting human endosialin expressing cells in vivo comprising administering the agent to the transgenic rodent comprising a nucleotide sequence encoding human endosialin integrated into the genome of the rodent, and measuring the level of detection of human endosialin expressing cells in the transgenic rodent by the agent and comparing the level of detection of human endosialin expressing cells in the transgenic rodent by the agent to the level of detection of human endosialin expressing cells in the transgenic rodent by a control. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Provided is a method for testing agents that modulate human endosialin expression in cells in vivo comprising measuring the expression of human endosialin in cells of the transgenic rodent comprising a nucleotide sequence encoding human endosialin integrated into the genome of the rodent, administering the agent to the transgenic rodent, measuring the expression of human endosialin in cells of the transgenic rodent after administration of the agent and comparing the level of expression of human endosialin in cells of the transgenic rodent after administration of the agent to the level of expression of human endosialin in cells of the transgenic rodent before administration of the agent. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for screening test agents that inhibit tumor growth comprising grafting a tumor onto the transgenic rodent comprising a nucleotide sequence encoding human endosialin integrated into the genome of the rodent, administering a test agent or a control agent to the transgenic rodent, measuring tumor growth in the transgenic rodent; and comparing tumor growth in the transgenic rodent administered with the test agent to tumor growth in the transgenic rodent administered with a control agent wherein the test agent is selected as an agent that inhibits tumor growth if the tumor growth in the transgenic rodent administered with the test agent is decreased with respect to tumor growth in the transgenic rodent administered with the control agent. In preferred embodiments the tumor is syngeneic to the transgenic rodent. In some preferred embodiments the tumor is allogeneic or xenogeneic to the transgenic rodent. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

Also provided is a method for screening test agents that inhibit tumor metastasis comprising introducing tumor cells into the transgenic rodent comprising a nucleotide sequence encoding human endosialin integrated into the genome of the rodent, administering a test agent or a control agent to the transgenic rodent, measuring tumor metastasis in the transgenic rodent and comparing tumor metastasis in the transgenic rodent administered with the test agent to tumor metastasis in the transgenic rodent administered with a control agent, wherein the test agent is selected as an agent that inhibits tumor metastasis if the tumor metastasis in the transgenic rodent administered with the test agent is decreased with respect to tumor metastasis in the transgenic rodent administered with the control agent. In preferred embodiments tumor metastasis is measured by quantifying in vivo bioluminescence of lung met cells employing the lung met model. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO: 5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

The invention also provides a method of using the rodents described herein to identify agents that can positively or negatively affect the expression and/or protein function of human endosialin within these rodents.

The invention further provides a method of using the HUE rodents to optimize the use of the agents for maximizing their exposure to stimulate or suppress a given human endosialin biological activity within these rodents.

### BRIEF DESCRIPTION OF THE DRAWINGS

FIG. 1 illustrates the development scheme of the human endosialin expressing, HUE, mice and generation of the human endosialin targeting vector. A 2274 bp fragment containing the human endosialin open reading frame (ORF) (SEQ ID NO: 3) was cloned into a fragment containing the mouse endosialin gene (SEQ ID NO: 1), which in turn disrupts the mouse endosialin gene function. This fragment was introduced into murine embryonic stem cells and selected for integration using neomycin selection. Selected clones were screened to identify those that integrated the construct into the mouse endosialin gene locus. Clones containing the targeting construct in the mouse endogenous endosialin locus were grown and introduced into mouse blastocysts and implanted into pseudo-pregnant recipient mice. Mouse pups were screened for targeting construct by PCR using mouse tail clipping. Those positive were then grown and backcrossed onto C57BL/6 strain mice. Heterozygotes were then re-crossed to generate HUE, homozygous murine endosialin null/human endosialin functional mice.

FIG. 2 shows expression of human endosialin within HUE mice. Expression of human endosialin in human pericytes and lung tissue derived from HUE mice. HUE mice or parental wild type C57BL/6 mice were harvested for lung tissue from naïve mice or mice derived from a cancer study whereby the mice have lung metastases. Pericytes were obtained from human peripheral blood. Proteins were extracted from cells or tissue and anti-human endosialin antibodies (MORAb-004) or control antibody (−009) were used to immunoprecipitate (IP) protein using standard IP methods. IP preps were then run on a SDS-PAGE gel and western blotted using the rabbit Rbt-TEM-1-55-2 antibody that can recognize human endosialin. As shown, HUE mice were positive for human endosialin protein in contrast to control parental mice which were negative.

FIG. 3 shows the utility of mice in studying the activity of human endosialin agents in a subcutaneous tumor model. Analytical mice were used to identify agents that can affect endosialin function in a SC model. HUE mice were generated to have tumor cells implanted subcutaneously. Once tumor began to grow, mice were treated for 5 days with 50 mg/kg of an antibody that can bind and suppress human endosialin activity in vitro (Tomkowicz et. al. (2007) Proc. Natl. Acad. Sci. USA 104:17965-17970). Mice were then screened and tumor measured for 17 days. As shown, HUE mice treated with anti-human endosialin antibody, but not control (CT) human IgG, had reduced tumor growth as compared to wild type C57 (wt C57) mice.

FIG. 4 shows the utility of HUE mice in studying the activity of human endosialin agents in a lung metastasis model. Analytical mice were used to identify agents that can affect endosialin function in a Lung met model. Lewis lung carcinoma (LLC) cells transduced with a luciferase gene were injected i.v. into HUE. Mice were treated every other day for 5 days, starting a day before tumor cell implantation and on day 3, 5, 7, and 10 post-implantation, with 50 mg/kg of FB5 or MORAb-004 that can bind and suppress human endosialin activity in vitro (Tomkowicz et. al. (2007) Proc. Natl. Acad. Sci. USA 104:17965-17970) or PBS control. The magnitude of metastatic disease in the lung was quantified by in vivo bioluminescence imaging three times a week up to day 14. Mice treated with either anti-human endosialin antibody showed reduced metastatic disease as compared to the vehicle-treated mice. MAb tx=antibody treatment regimen indicated by red arrowheads. *=P<0.05.

FIG. 5 shows micro-CT analysis demonstrating a decrease in micro-vascular density in anti-endosialin-treated tumor versus control-treated tumor. In the PBS-treated tumor control, the tumor volume is 2039 mm³, whereas the tumor volume of the MORAb-004—treated tumor is 637 mm³.

### DETAILED DESCRIPTION OF ILLUSTRATIVE EMBODIMENTS

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al. CURRENT PROTOCOLS IN MOLECULAR BIOLOGY, John Wiley & Sons, New York (1998); Sambrook et al. MOLECULAR CLONING: A LABORATORY MANUAL, 2D ED., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

Provided herein are methods for generating a human endosialin expressing rodents, preferably a mouse, whereby the endogenous endosialin gene is made non-functional. The human endosialin rodent, referred to as a HUE rodent, can be used to study the function of human endosialin in animal models as well as to develop agents that can affect human endosialin biological functions. The methods can be used to study the effect of the agents on normal endosialin physiology, or the effect of agents in disease models involving the endosialin pathway.

Without wishing to be bound by any particular theory of operation, it is believed that human endosialin positive cells (HEPCs) are precursors for cells that participate in the formation of vessels associated with neovascularization and neovascular disease such as cancer, inflammation and opthamalogic diseases. Therefore, HUE rodents may be useful in studying the effects of agents that can bind human endosialin protein or mRNA to study the role of endosialin in neovascular associated diseases. Thus, HUE rodents serve as ideal animal models to identify and develop human endosialin targeting agents for treating neovascular disease such as cancer and non-malignant pathologies. Conversely, HUE rodents serve as ideal animals to identify human endosialin targeting agents for promoting wound healing. HUE rodents are also useful for screening for active pharmacologic agents that can bind to human endosialin mRNA or protein including antibodies, endosialin binding proteins, vaccines, nucleic acid-based agents including antisense oligodeoxynucleotides and/or inhibitory micro RNAs, and viral-based gene therapies.

As used herein, the term “agent” or “targeting agent” refers to any molecule that can be used to inhibit or stimulate the activity of human endosialin, including but not limited to small chemical entities, RNAi, antisense oligonucleotides, agonists, natural ligands, antibodies, antigen-binding fragments of antibodies, endosialin binding proteins, vaccines and endosialin expressing whole cell vaccine. The term “targeting agent” or “agent” includes any molecule that binds to human endosialin that is bound to a molecule that stimulates or inhibits the activity of human endosialin.

As used herein, the term “endosialin-positive cell” (EPC) or “endosialin-expressing cell” refers to a cell that expresses endosialin. In some cases, the EPC is isolated from primary tissues or endothelial cultures, for example from HUE rodents described herein, using antibodies or binding proteins that can bind endosialin or endosialin-expressing progeny of such cells. EPCs may have the ability to form vessels.

As used herein, the term “inhibition of growth” means a decrease in the number of cells, in culture, by about 5%, preferably 10%, more preferably 20%, more preferably 30%, more preferably 40%, more preferably 50%, more preferably 60%, more preferably 70%, more preferably 80%, more preferably 90%, and most preferably 100%. In vivo inhibition of cell growth may be measured by assays known in the art. For example, “inhibition of growth” may be used to refer to the instance in which the tumor growth in a HUE rodent contacted with a test agent is reduced relative to the tumor growth in the absence of the test agent.

As used herein, the term “stimulation of growth” means an increase in the number of cells, in culture, by about 5%, preferably 10%, more preferably 20%, more preferably 30%, more preferably 40%, more preferably 50%, more preferably 60%, more preferably 70%, more preferably 80%, more preferably 90%, and most preferably 100%. In vivo stimulation of cell growth may be measured by assays known in the art. For example, “stimulation of growth” may be used to refer to the instance in which the tumor growth in a HUE rodent contacted with a test agent is increased relative to the tumor growth in the absence of the test agent.

The term “analysis” refers to analyzing RNA, protein or cellular profiles from HUE rodents. Analysis can be performed using any method used by those skilled in the art such as but not limited to differential expression methods such as microarray, cDNA libraries, SAGE, subtraction, or protein arrays.

The term “biological effect” refers to the inhibition or stimulation of a condition. A biological effect relieves to some extent one or more of the symptoms of the abnormal condition. In reference to the treatment of abnormal conditions, a biological effect can refer to one or more of the following: (a) an increase or decrease in the proliferation, growth, and/or differentiation of cells; (b) inhibition (i.e., slowing or stopping) of growth of tumor cells in vivo (c) promotion of cell death; (d) inhibition of degeneration; (e) relieving to some extent one or more of the symptoms associated with the abnormal condition; and (f) enhancing the function of a population of cells. Pharmacologic agents and derivatives thereof described herein effectuate the biological effect alone or in combination with conjugates or additional components of the compositions of the invention.

As used herein, the term “about” refers to an approximation of a stated value within an acceptable range. Preferably the range is +/−5% of the stated value.

As used herein, the term “normal tissue” refers to non-diseased tissue from mammalian embryos, fetal or adult sources.

As used herein, the term “diseased tissue” refers to tissue from mammalian embryos, fetal or adult sources that are dysplastic. Tissues can be from but not limited to malignant sources; disease of the eye; or disease from tissues with infection and/or inflammation.

As used herein, the term “endosialin ligand” refers to any protein or biochemical that can bind to cell surface endosialin.

As used herein, the term “targeting construct” or “targeting vector” refers to a vector having a recombinant DNA fragment containing the human endosialin open reading frame cloned in the sense orientation. According to the methods described herein, the nucleotide sequence encoding human endosialin integrates into a genomic fragment containing the native rodent endosialin gene, preferably functionally disrupting the rodent endosialin gene. In some embodiments, the targeting vector may contain a selection marker (e.g., a neomycin selection marker) for facilitating screening of recombinant cells.

As used herein, the term “analytical mice” or “analytical rodents” refers to HUE mice or rodents that can be used to identify agents such as antibodies, antigen-binding fragments thereof or binding proteins that can recognize endosialin expression on the cell surface or inhibit tumor growth or metastasis; agents such as antisense deoxynucleotides or inhibitory RNAs that can bind to human endosialin message in the cell in which these agents can be tested for the ability to affect endosialin pathway as it relates to disease and potentially be used to discover, optimize and/or validate the use of an agent for therapeutic use or diagnosis.

The term “functionally disrupted,” as used herein, refers to a gene that has a mutation that prevents the normal function of the gene, e.g., prevents expression of a gene of interest product or prevents expression of normal amounts a gene of interest product. The mutation causing the functional disruption can be an insertion, deletion or point mutation(s) which alters the amino acid sequence of the endogenous gene product encoded therein. The mutation causing the functional disruption may also be a replacement of the endogenous gene with a transgene (i.e, a “knock-in”).

The term “substantially reduced or null” is intended to mean that essentially undetectable amounts of normal endogenous gene product is produced in cells of the animal. This type of mutation is also referred to in the art as a “null mutation” and an animal carrying such a null mutation is also referred to as a “knockout animal.” This type of mutation can also be referred to as a “knock-in” when the endogenous gene is replaced with a transgene.

The term “transgenic rodent”, as used herein, refers to a rodent having cells that contain a transgene, wherein the transgene was introduced into the rodent or an ancestor of the rodent at a prenatal, e.g., an embryonic, stage. A “transgene” is a DNA which is integrated into the genome of a cell from which a transgenic rodent develops and which remains in the genome of the mature animal, thereby directing the expression of an encoded gene product in one or more cell types or tissues of the transgenic rodent. A type of transgenic rodent is the “knock-in,” where the endogenous gene is replaced with a transgene.

### Human Endosialin Expressing Rodents

Human endosialin rodents are rodents engineered to express a human endosialin gene from the rodent's endogenous endosialin gene locus. The endogenous endosialin gene of HUE rodents is preferably functionally disrupted or substantially reduced or null. The HUE rodent therefore can be used to screen, develop and study agents that can have a direct effect on human endosialin in suppressing or promoting biological pathways associated with development and/or disease. These rodents provide unique models to study the function of human endosialin in light of the fact that rodent endosialin is functionally disrupted or substantially reduced or null by the introduction of the human gene into the functional region of the gene encoding for the rodent endosialin. This feature avoids any potential redundancy that can occur if rodent endosialin is present which may be able to support a disease state or development process. Traditional transgenic mice that contain a human endosialin gene and an intact rodent endosialin gene may diminish the ability to clearly identify agents that can bind human endosialin to elucidate the agent's pharmacologic activity on suppression or activation of human endosialin gene function in supporting a disease state.

In preferred embodiments the rodent is a mouse. Preferred mice of the invention are those in which various mouse strains are used for disease models. These strains include but are not limited to DBA2, C57BL/6, and SJ123 strains. The mice are rendered deficient for endogenous mouse endosialin but have a functional human endosialin gene product.

Preferred transgenic rodents described herein are made by homologous recombination of a targeted native or endogenous allele encoding endosialin with a transgene comprising a sequence encoding human endosialin flanked by flanking sequences which effect the homologous recombination of the transgene with the native allele, wherein the expression of the human endosialin is under the control of native gene expression regulatory sequences of the native allele. In preferred embodiments, at least a portion of the native endosialin allele is replaced with the human endosialin sequence.

HUE rodents are engineered to express human endosialin and lack endogenous endosialin gene product. In some preferred embodiments, human complementary cDNA encoding the entire endosialin open reading frame (ORF) is cloned into a rodent genomic fragment containing the rodent endosialin gene locus. The targeting construct may contain a selectable marker (e.g., a neomycin resistance gene) to aid in selection of cells that uptake the construct. The resultant construct is then linearized and introduced in a rodent embryonic stem cell (ESC) line, and screened via polymerase chain reaction (PCR) for the integration of the construct into the rodent endosialin locus by using standard methods known to those skilled in the art of generating gene knock out or transgenic embryonic stem (ES) cells. Clones found to have the targeting construct integrated into the endogenous rodent endosialin gene locus are expanded and further validated by southern blot. A clone is used to inject into rodent blastocysts and resultant rodent pups are screened using methods known to those skilled in the art, including genomic PCR or southern blotting using DNA extracted from tail clippings to identify those rodents containing the targeting construct. These chimeric rodents are then grown and crossed to non-transgenic rodents to generate heterozygotes and ultimately homozygous rodents containing null rodent endosialin and functional human endosialin expression.

Also provided are isolated genetic knock-in cells. In some embodiments, the isolated knock-in cell is a genetic knock-in primary cell obtained from a knock-in rodent as described above. In some embodiments, the isolated knock-in cell is a clone of, is genetically identical to, or, preferably is progeny of a genetic knock-in primary cell obtained from a knock-in rodent as described above.

### Antibodies, Binding Proteins, Nucleic Acid Agents.

The antibodies and binding proteins of the invention specifically bind endosialin on the cell surface within or derived from HUE rodents. In some embodiments, the antibodies and proteins bind to cells expressing human endosialin within normal tissue. In other embodiments, the antibodies and proteins bind to cells in diseased tissue. In yet other embodiments, the antibodies and proteins bind to cells in primary cultures derived from HUE rodents. In some embodiments, the antibody or binding protein is bound to a molecule that inhibits or stimulates human endosialin or to a detectable label.

Preferred antibodies or antigen-binding fragments thereof and proteins suitable for use in the method of the invention, include, for example, fully human antibodies, human antibody homologs, humanized antibody homologs, chimeric antibody homologs, Fab, Fab′, F(ab′)₂ and F(v) antibody fragments, single chain antibodies, and monomers or dimers of antibody heavy or light chains, endosialin ligands or mixtures thereof that can isolate the cells from tissue or culture.

The antibodies may include intact immunoglobulins of any isotype including types IgA, IgG, IgE, IgD, IgM (as well as subtypes thereof). The light chains of the immunoglobulin may be kappa or lambda.

The antibody-binding fragments of antibodies include portions of intact antibodies that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. Thus, antigen binding fragments, as well as full-length dimeric or trimeric polypeptides derived from the above-described antibodies are themselves useful.

The antibodies, antigen-binding fragments, or endosialin binding proteins may be used alone or as a conjugate. Conjugation of antibodies and binding proteins is well-known in the literature.

The antibodies and antigen-binding fragments thereof include derivatives that are modified, e.g., by the covalent attachment of any type of molecule to the antibody such that covalent attachment does not prevent the antibody from binding to its epitope. Examples of suitable derivatives include, but are not limited to glycosylated antibodies and fragments, acetylated antibodies and fragments, pegylated antibodies and fragments, phosphorylated antibodies and fragments, and amidated antibodies and fragments. The antibodies and derivatives thereof of the invention may themselves by derivatized by known protecting/blocking groups, proteolytic cleavage, linkage to a cellular ligand or other proteins, and the like. Further, the antibodies and derivatives thereof of the invention may contain one or more non-classical amino acids.

Polyclonal antibodies such as those derived from peripheral blood of mammalian cell hosts also may be used. Such cells may be fused with myeloma cells, for example to form hybridoma cells producing antibodies that can bind to human endosialin in vivo.

Without wishing to be bound by any particular theory of operation, it is believed that the antibodies or antigen-binding fragments thereof are particularly useful to bind endosialin expressing tissues and cells within HUE rodents due to a binding of the antibody or antigen-binding fragment thereof to an extracellular epitope. This leads to a decrease in the dissociation (K_{d}) of the antibody. This is an especially good feature for targeting cells for isolation from tissue or tissue culture.

U.S. Publ. No. 2008-0248034 describing antibodies that bind human endosialin is incorporated by reference herein. In some embodiments, the agent is a monoclonal antibody that binds human endosialin or an antigen binding fragment thereof. In preferred embodiments, the agent is a humanized monoclonal antibody that binds endosialin, or an antigen binding fragment thereof. Preferably, the antibody or antigen-binding fragment comprises a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively. Most preferably, the agent is a humanized monoclonal antibody comprising a heavy chain comprising CDR1, CDR2, and CDR3 of SEQ ID NO:5, 6, and 7, respectively, and a light chain comprising CDR1, CDR2, and CDR3 of SEQ ID NO: 8, 9, and 10, respectively, or an antigen binding fragment thereof. In one embodiment, the agent is MORAb-004 (Morphotek Inc., Exton, Pa.).

SEQ ID NO: 5
Gly Tyr Thr Phe Thr Asp Tyr Val Ile His
SEQ ID NO: 6:
Tyr Ile Asn Pro Tyr Asp Asp Asp Thr Thr Tyr Asn
Gln Lys Phe Lys Gly
SEQ ID NO: 7:
Ala Arg Arg Gly Asn Ser Tyr Asp Gly Tyr Phe Asp
Tyr Ser Met Asp Tyr
SEQ ID NO: 8:
Arg Ala Ser Gln Asn Val Gly ThrAla Val Ala
SEQ ID NO: 9:
Ser Ala Ser Asn Arg Tyr Thr
SEQ ID NO: 10:
Gln Gln Tyr Thr Asn Tyr Pro Met Tyr Thr

Nucleic acids agents of the invention are those that can be administered via intravenous, intramuscular, aerosolized, topical or oral routes to inhibit or suppress the in vivo expression of human endosialin. Nucleic acids include antisense deoxynulceotides, siRNA and miRNA, as well as derivatives thereof. Not wanting to be limited on use, however, the development of nucleic acid agents that work in vivo is not obvious using in vitro based systems. Therefore the use of HUE rodents to screen for nucleic acid agents enables the development of the agents for potential pharmacologic use. Nucleic acid agents may be used alone or as a conjugate. Conjugation of nucleic acids is well-known in the literature.

Nucleic acid screening includes the effect of the agents in suppressing human endosialin expression in positive normal or diseased tissues whereby analysis is done measuring steady state mRNA, protein and/or endosialin expressing cells derived from exposed HUE rodents.

The analytical rodents of the invention include rodents that are prepared to exhibit a disease state such as but not limited to cancer, inflammatory or opthalmologic disease whereby human endosialin agents can be tested for altering the disease state. Conversely, analytical rodents can be used in models where defective or suboptimal endosialin activity exists such as but not limited to wound healing whereby the agents can be screened to identify those capable of stimulating a defective biological process via human endosialin activity.

The invention also encompasses the use of HUE rodents to identify agents that can be used as diagnostic probes to screen for the presence or absence of disease. Diagnostic probes include molecular entities that are used by those skilled in the art, including but not limited to monoclonal antibodies, binding proteins and labeled complementary nucleic acids to the human endosialin gene product.

### Methods of Screening for Human Endosialin Binding Agents Via HUE Rodents.

### Screening for Antibody or Binding Protein Specificity

Screening for antibodies, antigen-binding fragments, or endosialin-binding proteins that specifically bind to cells or tissues expressing human endosialin within HUE rodents or cells, tissues, or fluids derived therefrom may be accomplished using immunoassay techniques known in the art, e.g., immunohistochemistry (IHC) or immunocytochemistry (ICC) in tissues or cells that express endosialin. Conversely, antibodies, antigen-binding fragments, or endosialin binding proteins may be labeled with a detectable label (e.g., radioactive isotope, chromophoric fluorescent dye, chemiluminescent compound, phosphorescent compound, biotin or an enzyme) and used for detection of endosialin-expressing cells, e.g., in in vivo imaging within HUE rodents or tissues thereof. Positive binding antibodies or binding proteins identified from analytical mice can be further screened for reactivity in a cell ELISA-based assay to identify agents that can bind human endosialin expressing cells derived from tissues of HUE rodents. Clones that produce antibodies or binding proteins that are reactive to human endosialin are selected for further expansion and development.

The antibodies, derivatives and binding proteins thereof of the invention have binding affinities that include a dissociation constant (Kd) of less than 1×10⁻². In some embodiments, the Kd is less than 1×10⁻³. In other embodiments, the Kd is less than 1×10⁻⁴. In some embodiments, the Kd is less than 1×10⁻⁵. In still other embodiments, the Kd is less than 1×10⁻⁶. In other embodiments, the Kd is less than 1×10⁻⁷. In other embodiments, the Kd is less than 1×10⁻⁸. In other embodiments, the Kd is less than 1×10⁻⁹. In other embodiments, the Kd is less than 1×10⁻¹⁰. In still other embodiments, the Kd is less than 1×10⁻¹¹. In some embodiments, the Kd is less than 1×10⁻¹². In other embodiments, the Kd is less than 1×10⁻¹³. In other embodiments, the Kd is less than 1×10⁻¹⁴. In still other embodiments, the Kd is less than 1×10⁻¹⁵.

### Methods of Employing HUE Rodents to Screen for Active Agents.

The methods of the invention are suitable for use in disease and non-disease states.

The invention is suitable for screening of agents in HUE rodents that exhibit a dysplastic disorder that is marked by increased expression of human endosialin in the diseased tissue in relation to normal tissues. Malignant tissues that may be studied for anti-human endosialin activity include but are not limited to ovarian tumors, renal tumors, colorectal tumors, pancreatic tumors, prostate tumors lung tumors, fallopian tube tumors, uterine tumors, and brain tumors. Tissues affected with inflammation may also be studied which include but are not limited to arthritic tissue or tissue from inflamed airways. Eye tissue may also be studied for anti-endosialin activity in the mice. Conversely, disease states that can benefit from enhanced endosialin activity can be used to screen for active agents that enhance endosialin biological activity. These can be agents that act as endosialin ligand agonists as well as agents that modify endosialin protein structure to enhance it endogenous activity(s). Recently, it has been demonstrated that endosialin pathway crosstalks with the platelet derived growth factor (PDGF) receptor pathway to promote biological activities associated with neovascularization (Tomkowicz, B., et. al. (2010) Cancer Biol. Ther. 9:1-8). Endosialin binding agents that can promote activity of PDGF receptor pathway may offer benefit to wound healing and other related disorders.

Endosialin binding proteins as well as anti-endosialin antibodies and antigen binding fragments thereof may be used to study pharmacologic activity in HUE rodents. These activities include maximizing therapeutic activity and/or studying effects of agents in toxicologic studies. The anti-endosialin antibodies and antigen binding fragments may be used to study dosing effects and drug concentration for maximizing anti-endosialin activity.

The invention is also suitable for screening for agents that can suppress disease using HUE rodents. These agents may include an angiogenesis inhibitor molecule that modulates an endosialin protein. The methods comprise contacting an HUE rodent bearing a tumor with a test molecule and detecting the inhibition of the tumor growth, wherein the test molecule is identified as an angiogenesis inhibitor molecule that modulates the endosialin protein or activity when the tumor growth in the mouse contacted with the test molecule is reduced relative to the tumor growth in the mouse not contacted by the test molecule.

Any suitable tumor can be injected in any suitable manner to provide a model for the testing of anti-angiogenic molecules. The rodent recipient of the tumor can be any suitable strain. The tumor can be syngeneic, allogeneic, or xenogeneic to the transgenic rodent. The recipient can be immunocompetent or immunocompromised in one or more immune-related functions, included but not limited to nu/nu, scid and beige mice. The HUE rodents can be inoculated with syngeneic tumor cells, e.g. B16 melanoma (ATCC). The effect of antibody or antigen-binding fragment thereof administration on tumor growth can be ascertained by quantizing the primary or metastatic tumor growth using conventional methods.

A variety of different test anti-angiogenic molecules may be identified using the method as provided herein. Anti-angiogenic molecules can encompass numerous chemical classes. In certain embodiments, they are organic molecules, preferably small organic compounds having a molecular weight of more than 50 and less than about 2,500 daltons. Anti-angiogenic molecules can comprise functional groups necessary for structural interaction with proteins, particularly hydrogen bonding, and may include at least an amine, carbonyl, hydroxyl or carboxyl group, preferably at least two of the functional chemical groups. The anti-angiogenic molecules can comprise cyclical carbon or heterocyclic structures and/or aromatic or polyaromatic structures substituted with one or more of the above functional groups. Anti-angiogenic molecules also include biomolecules like peptides, saccharides, fatty acids, steroids, purines, pyrimidines, derivatives, structural analogs or combinations thereof. Test anti-angiogenic molecules of interest also can include peptide and protein agents, such as antibodies or binding fragments or mimetics thereof, e.g., Fv, F(ab′)₂ and Fab.

Test anti-angiogenic molecules also can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

A test anti-angiogenic molecule is identified as an inhibitor when it is capable of specifically inhibiting the growth of a tumor by at least 20%, often 30, 40, 50, 60, 70, 80 or 90%, and sometimes 100%. The growth inhibition can be quantified using any convenient method of measurement. For example, for primary tumor growth, perpendicular measurements are taken of the tumor mass to calculate tumor volume. Metastatic growth can be ascertained by microscopic or macroscopic analysis, as appropriate. The tumor can be syngeneic, allogeneic, or xenogeneic to the transgenic animal. The test molecules can be administered at the time of tumor inoculation, after the establishment of primate tumor growth, or after the establishment of local and/or distant metastases. Single or multiple administration of the test molecule can be given using any convenient mode of administration, including but not limited to intravenous, intraperitoneal, intratumoral, subcutaneous, and intradermal.

The following Examples are provided to illustrate the present invention, and should not be construed as limiting thereof.

### EXAMPLES

### Example 1

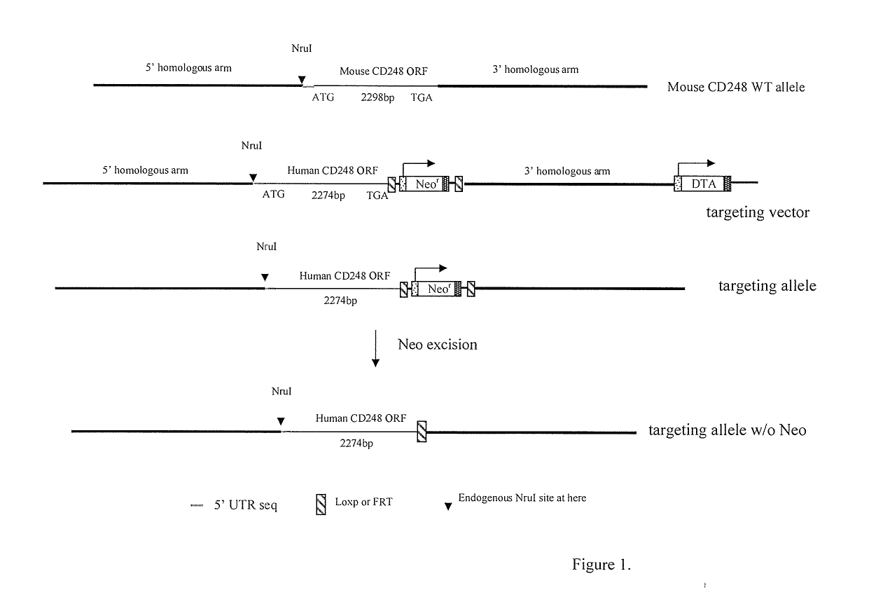

HUE mice were engineered to express human endosialin and lack endogenous endosialin gene product. A human complementary cDNA encoding the entire endosialin open reading frame (ORF) was cloned into a mouse genomic fragment containing the mouse endosialin gene locus. FIG. 1 provides the strategy for cloning the locus. The targeting construct contains a neomycin resistance gene to aid in selection of cells that incorporate the construct. The mouse chromosome 19 sequence (n.t.#5,038,078-5,100,640) was retrieved from the Ensembl database and used as reference. The 5′ homology arm (5.0 kb) and 3′ homologous arm (2.8 kb) was generated by PCR from mouse RP23-410O10 BAC.

The homology arms and the knock-in region of the targeting vector were created by PCR using the following primers and conditions.

5′ arm:
Primers:
5′ arm forward:
(SEQ ID NO: 12)
5′-acacacgcgtGCTTCAAGTCTCAGCCCTGCCA-3′
5′ arm reverse:
(SEQ ID NO: 13)
5′-acaacatcgcgaCGCCCCCCGACTGG-3′
Template: RP23-410O10 BAC
3′ arm:
Primers:
3′ arm forward:
(SEQ ID NO: 14)
5′-cacagtcgacGTGCAGATGCCCCTTTGTGGGA-3′
3′ arm reverse:
(SEQ ID NO: 15)
5′-cacggatccGGTAAGAGGGTTAGAGCGTATGC-3′
Template: RP23-410010 BAC
hCD248 knock-in region
Primers: Human CD248 forward:
(SEQ ID NO: 16)
5′-gggcgtcgcgaTGCTGCTGCGCCTGTTGCTGG-3′
Human CD248 reverse:
(SEQ ID NO: 17)
5′-caaaggcgcgccCCCATCACACGCTGGTTCTGCAGGTCTG-3′

Capital letters represent sequences belonging to the arms. The lower case letters represent restriction endonuclease sites added for subsequent cloning steps. PCR cycling conditions were as follows: 94° C. 15 sec, 58° C. 15 sec, 68° C. 6 min, 25 cycles.

The knock-in region contained the human CD248 cDNA plasmid. These fragments were cloned in the LoxNwCD, and were confirmed by restriction digestion and end-sequencing. The final vector was obtained by standard molecular cloning. Aside from the homology arms and the knock-in human CD248 fragment, the final vector also contains loxP sequences flanking the Neo expression cassette (for positive selection of the ES cells), and a DTA expression cassette (for negative selection of the ES cells). The final vector was confirmed by both restriction digestion and end sequencing analysis. The human endosialin targeting sequence is as follows (SEQ ID NO: 11) (homology arms in italics; knock-in region in bold; LoxP sites underlined; Confirmed sequences highlighted/shaded):

```
      1 CTAGTGAGTC GTATTACGTA GCTTGGCGTA ATCATGGTCA TAGCTGTTTC CTGTGTGAAA
    61 TTGTTATCCG CTCACAATTC CACACAACAT ACGAGCCGGA AGCATAAAGT GTAAAGCCTG
    121 GGGTGCCTAA TGAGTGAGCT AACTCACATT ACATGTGAGC AAAAGGCCAG CAAAAGGCCA
    181 GGAACCGTAA AAAGGCCGCG TTGCTGGCGT TTTTCCATAG GCTCCGCCCC CCTGACGAGC
    241 ATCACAAAAA TCGACGCTCA AGTCAGAGGT GGCGAAACCC GACAGGACTA TAAAGATACC
    301 AGGCGTTTCC CCCTGGAAGC TCCCTCGTGC GCTCTCCTGT TCCGACCCTG CCGCTTACCG
    361 GATACCTGTC CGCCTTTCTC CCTTCGGGAA GCGTGGCGCT TTCTCATAGC TCACGCTGTA
    421 GGTATCTCAG TTCGGTGTAG GTCGTTCGCT CCAAGCTGGG CTGTGTGCAC GAACCCCCCG
    481 TTCAGCCCGA CCGCTGCGCC TTATCCGGTA ACTATCGTCT TGAGTCCAAC CCGGTAAGAC
    541 ACGACTTATC GCCACTGGCA GCAGCCACTG GTAACAGGAT TAGCAGAGCG AGGTATGTAG
    601 GCGGTGCTAC AGAGTTCTTG AAGTGGTGGC CTAACTACGG CTACACTAGA AGAACAGTAT
    661 TTGGTATCTG CGCTCTGCTG AAGCCAGTTA CCTTCGGAAA AAGAGTTGGT AGCTCTTGAT
    721 CCGGCAAACA AACCACCGCT GGTAGCGGTG GTTTTTTTGT TTGCAAGCAG CAGATTACGC
    781 GCAGAAAAAA AGGATCTCAA GAAGATCCTT TGATCTTTTC TACGGGGTCT GACGCTCAGT
    841 GGAACGAAAA CTCACGTTAA GGGATTTTGG TCATGAGATT ATCAAAAAGG ATCTTCACCT
    901 AGATCCTTTT ACGCGCCCTG TAGCGGCGCA TTAAGCGCGG CGGGTGTGGT GGTTACGCGC
    961 AGCGTGACCG CTACACTTGC CAGCGCCCTA GCGCCCGCTC CTTTCGCTTT CTTCCCTTCC
  1021 TTTCTCGCCA CGTTCGCTTT CCCCGTCAAG CTCTAAATCG GGGGCTCCCT TTAGGGTTCC
  1081 GATTTAGTGC TTTACGGCAC CTCGACCCCA AAAAACTTGA TTTGGGTGAT GGTTCACGTA
  1141 GTGGGCCATC GCCCTGATAG ACGGTTTTTC GCCCTTTGAC GTTGGAGTCC ACGTTCTTTA
  1201 ATAGTGGACT CTTGTTCCAA ACTGGAACAA CACTCAACCC TATCTCGGGC TATTCTTTTG
  1261 ATTTATAAGG GATTTTGCCG ATTTCGGCCT ATTGGTTAAA AAATGAGCTG ATTTAACAAA
  1321 AATTTAACGC GAATTTTAAC AAAATATTAA CGTTTACAAT TTAAATATTT GCTTATACAA
  1381 TCTTCCTGTT TTTGGGGCTT TTCTGATTAT CAACCGGGGT AAATCAATCT AAAGTATATA
  1441 TGAGTAAACT TGGTCTGACA GTTACCAATG CTTAATCAGT GAGGCACCTA TCTCAGCGAT
  1501 CTGTCTATTT CGTTCATCCA TAGTTGCCTG ACTCCCCGTC GTGTAGATAA CTACGATACG
  1561 GGAGGGCTTA CCATCTGGCC CCAGTGCTGC AATGATACCG CGAGACCCAC GCTCACCGGC
  1621 TCCAGATTTA TCAGCAATAA ACCAGCCAGC CGGAAGGGCC GAGCGCAGAA GTGGTCCTGC
  1681 AACTTTATCC GCCTCCATCC AGTCTATTAA TTGTTGCCGG GAAGCTAGAG TAAGTAGTTC
  1741 GCCAGTTAAT AGTTTGCGCA ACGTTGTTGC CATTGCTACA GGCATCGTGG TGTCACGCTC
  1801 GTCGTTTGGT ATGGCTTCAT TCAGCTCCGG TTCCCAACGA TCAAGGCGAG TTACATGATC
  1861 CCCCATGTTG TGCAAAAAAG CGGTTAGCTC CTTCGGTCCT CCGATCGTTG TCAGAAGTAA
  1921 GTTGGCCGCA GTGTTATCAC TCATGGTTAT GGCAGCACTG CATAATTCTC TTACTGTCAT
  1981 GCCATCCGTA AGATGCTTTT CTGTGACTGG TGAGTACTCA ACCAAGTCAT TCTGAGAATA
  2041 GTGTATGCGG CGACCGAGTT GCTCTTGCCC GGCGTCAACA CGGGATAATA CCGCGCCACA
  2101 TAGCAGAACT TTAAAAGTGC TCATCATTGG AGAACGTTCT TCGGGGCGAA AACTCTCAAG
  2161 GATCTTACCG CTGTTGAGAT CCAGTTCGAT GTAACCCACT CGTGCACCCA ACTGATCTTC
  2221 AGCATCTTTT ACTTTCACCA GCGTTTCTGG GTGAGCAAAA ACAGGAAGGC AAAATGCCGC
  2281 AAAAAAGGGA ATAAGGGCGA CACGGAAATG TTGAATACTC ATACTCTTCC TTTTTCAATA
  2341 TTATTGAAGC ATTTATCAGG GTTATTGTCT CATGAGCGGA TACATATTTG AATGTATTTA
  2401 GAAAAATAAA CAAATAGGGG TTCCGCGCAC ATTTCCCCGA AAAGTGCCAC CTGACGTAGT
  2461 TAACAAAAAA AAGCCCGCCG AAGCGGGCTT TATTACCAAG CGAAGCGCCA TTCGCCATTC
  2521 AGGCTGCGCA ACTGTTGGGA AGGGCGATCG GTGCGGGCCT CTTCGCTATT ACGCCAGCTG
  2581 GCGAAAGGGG GATGTGCTGC AAGGCGATTA AGTTGGGTAA CGCCAGGGTT TTCCCAGTCA
  2641 CGACGTTGTA AAACGACGGC CAGTCCGTAA TACGACTCAC TTAAGGCGTA GTACGGGCCC
  2701 CCTCGGTCCG CTCTAGAACT ACGATCCAGA CATGATAAGA TACATTGATG AGTTTGGACA
  2761 AACCACAACT AGAATGCAGT GAAAAAAATG CTTTATTTGT GAAATTTGTG ATGCTATTGC
  2821 TTTATTTGTA ACCATTATAA GCTGCAATAA ACAAGTTAGA TCCTAGTGGA TCTGCATTCC
  2881 ACCACTGCTC CCATTCATCA GTTCCATAGG TTGGAATCTA AAATACACAA ACAATTAGGA
  2941 ATCAGTAGTT TAACACATTA TACACTTAAA AATTTTATAT TTACCTTAGA GCTTTAAATC
  3001 TCTGTAGGTA GTTTGTCCAA TTATGTCACA CCACAGAAGT AAGGTTTCCT TCACAAAGAG
  3061 ATCGCCTGAC ACGATTTCCT GCACAGGCTT GAGCCATATA CTCATACATC GCATCTTGGC
  3121 CACGTTTTCC ACGGGTTTCA AAATTAATCT CAAGTTCTAC GCTTAACGCT TTCGCCTGTT
  3181 CCCAGTTATT AATATATTCA ACGCTAGAAC TCCCCTCAGC GAAGGGAAGG CTGAGCACTA
  3241 CACGCGAAGC ACCATCACCG AACCTTTTGA TAAACTCTTC CGTTCCGACT TGCTCCATCA
  3301 ACGGTTCAGT GAGACTTAAA CCTAACTCTT TCTTAATAGT TTCGGCATTA TCCACTTTTA
  3361 GTGCGAGAAC CTTCGTCAGT CCTGGATACG TCACTTTGAC CACGCCTCCA GCTTTTCCAG
  3421 AGAGCGGGTT TTCATTATCT ACAGAGTATC CCGCAGCGTC GTATTTATTG TCGGTACTAT
  3481 AAAACCCTTT CCAATCATCG TCATAATTTC CTTGTGTACC AGATTTTGGC TTTTGTATAC
  3541 CTTTTTGAAT GGAATCTACA TAACCAGGTT TAGTCCCGTG GTACGAAGAA AAGTTTTCCA
  3601 TCACAAAAGA TTTAGAAGAA TCAACAACAT CATCAGGGTC CATGGCGAGG ACCTGCAGGT
  3661 CGCAGCCGCC CGCCGCGCGC TTCGCTTTTT ATAGGGCCGC CGCCGCCGCC GCCTCGCCAT
  3721 AAAAGGAAAC TTTCGGAGCG CGCCGCTCTG ATTGGCTGCC GCCGCACCTC TCCGCCTCGC
  3781 CCCGCCCCGC CCCTCGCCCC GCCCCGCCCC GCCTGGCGCG CGCCCCCCCC CCCCCCCCGC
  3841 CCCCATCGCT GCACAAAATA ATTAAAAAAT AAATAAATAC AAAATTGGGG GTGGGGAGGG
  3901 GGGGGAGATG GGGAGAGTGA AGCAGAACGT GGGGCTCACC TCGACCATGG TAATAGCGAT
  3961 GACTAATACG TAGATGTACT GCCAAGTAGG AAAGTCCCAT AAGGTCATGT ACTGGGCATA
  4021 ATGCCAGGCG GGCCATTTAC CGTCATTGAC GTCAATAGGG GGCGTACTTG GCATATGATA
  4081 CACTTGATGT ACTGCCAAGT GGGCAGTTTA CCGTAAATAC TCCACCCATT GACGTCAATG
  4141 GAAAGTCCCT ATTGGCGTTA CTATGGGAAC ATACGTCATT ATTGACGTCA ATGGGCGGGG
  4201 GTCGTTGGGC GGTCAGCCAG GCGGGCCATT TACCGTAAGT TATGTAACGC GGAACTCCAT

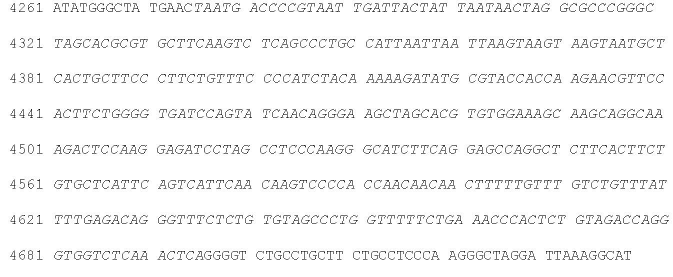

  4741 GCACCACCAT GTTGAGTTCT TTTGTTTGTT TTTTGTTTTG TTTTTAAAGA TTTATGTATT
  4801 TTATGTATGT GAGTACACTG TTGCTGTCTT CAGACACACC AGAAGAGGGC ATCAGATCCC
  4861 ATTACAGATG GTTGTGAGCC ACCATGTGGT TGCTGGGAAT TGATCTCAGG ACCTCTGGAA
  4921 GAGCAGTCAG TGCTCTTAAC AACTGAGCCA TCTCTCCAGC CCATTGTGTT TTGTTTTATT
  4981 GTTTTTGTTT TGTTTTGTTT TGTTTTGTTT TGTTTTGTTT TGTTTTAATA GAGTCTCATT
  5041 TATCTTTTTA CATCCCAGCC TGGCCTGAAA GTCACTATGC ATCTAAGGGT CACTCTAACT
  5101 TTCTATTCCT CTGTCTCCAA CTCTGAAGTG CTGGGATTTC AGGCACTTCT TACTAATTCT
  5161 TACTTACAGT TCTTTTGTTT CAGTTCTTTT GGTTTGGTTT TTTTTTTTTT TTTTTTTTTT
  5221 TTTCGAGACA GGGTTTCTCT GTGTAGCCCT GGCTGTCCTG GAACTCACTC TGTAGACCAG
  5281 GCTGTCCTCG AACTCAGCAA TCTGCCTGCC TTTGCCTCCC AAGTGCTGGG ATTTAAAGAC
  5341 GTGTGCCACC ACTGCCCGGC TCTTACTTAC AGTTCTTACT AAGCAAAGGC TCTAGGCCCA
  5401 GAGGCTCCAA GGCACAGCAG GGCTCACTGT GAGTCTGCTG GAGCAGTCTC CCAAGATGGG
  5461 AGTTCTCCAC TGGGCACAGG ATACCTGGCA ATAACTGGAG ATATGTTTTT GTTGTCCTAA
  5521 CTAGAAGGAT GCTATTCACA TATGGGTCAA AGCCAGGGCA CAGATCAAAC CACTCATAAC
  5581 TATATGTCAC TGAATGGCAA TAATGCCAGC TAGGATTGAG GGATTCTGGG TAGGAGTCAG
  5641 GTCATAAGGG GGTTCAAGTC AACAACAAAG CATATACCAT GGCAGAGGGA ACTGGTGAGA
  5701 GGTGGGAGGT AGGGGGACCT CAGAGAGAGG AGAGTCATGT GTGGGGGCTC AGGGACTATT
  5761 CTGCTTCAAC TGAGACCCAA GAAAGTGAAA GGCAAAGGGG GAGGGCATTT GGATGTTGCA
  5821 GGAAGGAGAG AACATGGGAT GAAAGCTCAG TGTCTGGTGG GCCTAAGGCT TGACACAGAC
  5881 ACTGCTAGAA CAAACAGAAG GACCAAGGGA GAAAGGGAGG GGAGGGCTGG CTGAGCGTTA
  5941 GAGACAGATC ACATAGCAGG ACTTTGCTTG CCTCTGAGTG AGACATGGCC AGAGAGGGTC
  6001 GTGAACAGAG GTGGCTAGAT CTGGCCAGGT CTGCTTTTTC TTTGTGTGTA CGTGGGCAAG
  6061 ATTATTTTAT TTTATCAGCG TTTTTGCCTG CATATACTGT ATGCACTACA TGTGTGCCCA
  6121 GTGTATGGAA GGGGTCAGAA GAGGACATCA GAGTCCCTGG AACTGGAGTT CCAGATGGTT
  6181 ATGAGCCTCC GTGTGGGTAA CAGGAGCCTA GCCCTGGCTC TTTGCAAGAG CAGCAAGTAC
  6241 TCTTGACCAC TGAGACAGCT CTCCAGCCCT CTTGTTTTTT AACGGTTCTC CCCATAAGGG
  6301 AGAAAAGGAG GAAGAGGAGT GGGGGGCAGG GTCCTAACTA GGAGATGGAG TGTGACCGCG
  6361 GTGGACAGAA AGGCAAAGGT GGTGCTGTGA AGTGGTCACA CTCTTGATCT AGTTTTGAAA
  6421 ACGATGCCAA CAGGATTTGC TGAGATACTG GACCAGGGGT GTGGGAGAGA GAGAAGGATG
  6481 AGCTCAAGCT TTGGCTCTGC TCCAGCTGAG GGATGATGGC CTCTGCTCTC CTGAGAAGGG
  6541 GCGTCGCCCT GCATGCTTTG AGCATAAGAC TTATGCTCAA AGGCAAGTGC TGTATAAACA
  6601 GCAGTTCTCA CAGCCTGGCT GTGTCTGCAG TGCCGACCTG GGCACTGTGA GGAAGTTAGT
  6661 GAAAGCACCC GGGTCCCCAC TGGCATCCAG ATTTGCTCAG GAGACTACCA AACCTTGGTC
  6721 CTGTACAAAA CATATATGCA AACAGGGAGG GCTATAACAC AATAGCGGGC TGAAGAGTCC
  6781 GCACACATGT ATCCCCATGT TGTGGAGGGG GACAATGGAA ACCTGGCAAG GTCACCTACA
  6841 CCTATAATCT CAGTACTGCT GCACAGAGAC CTACACAAAA GGGAAGAGGA GGCAGGAAAA
  6901 GAGAAGAGAA AGGGAGGGAA GGGAAGTGGA AAGGATTGAA ATAATGAAGA GGAGAAAGAA
  6961 GCGTGTGCGT GCTCGTGTGT GCGTGCATGC GTGAGTGTGT ATCCTAGCAC TTGGGAAGCA
  7021 GAGGCATTTG GATCTCTGTG AGCTCCAGAC CAGCCAGAAC TACATGGTCT AAACTACATA
  7081 GTGTCTTTAA AAAGGGCTGG GGTGTATGGG GGTGGGACAT GAACGAGGAC TCAGAGAGCC
  7141 TCGAACTCAC ACAGGTTAGT AGCAGAGTAT CCAGGCCAGG ACAGTGTTCC AAAGAACAAA
  7201 CCTTCTCTTT CACTTTCTCA TCCATGGGTC ATGAGATTTT TAGTGCTGGG AATTCAACCC
  7261 AGGACCAGAC ATCACCCTAC CACGAAGCCA CATCCCCAGC TGGCTCAGAA TCTGCGGAGA
  7321 ATATAAAGGA TCGTGTGTTA ATGCCAGACT CAGAGGTGAT GGGAAGACCA GGACTTTAAG
  7381 GTCATCCTGG ACCACACAGT GAGCTCCAGC TTAGCCTGGG CAACATGTAA GACTTTGCCT
  7441 CAAAAAATCA AAAACGGACA GGGCGTAGTG GTGCATGCCT TTAATCCCAG CACTCGGGAG
  7501 GCAGAAGCAG GCGGATTTCT GAGTTCGAAG CCATCCTGGT CTACAGAGTG AGTTCCAGGA
  7561 CAGCCAAGGC TATACAGAGA AACCCTGTCT CGGAAAAATA AATAAATAAA AAACGGGCTG
  7621 GAGTGATGCC TAAGCAGCGA AGAGTACTTC TTGCTCTTGC AGAGGACCCA AGTTTGGTTC
  7681 CCAGCACCCA CACGGTAGTT TAAAAATACC CGTAACTCCA GTTCCAGGGA ATTTGATGCC
  7741 CTCTTCTGAC TTCTGAAGGT AACAGGCACA CACACGGTAC ACACGCATAC ATGCAGGAAA
  7801 AACACTCATA AACAGAAAGC AAAAATGTTT TTAAAAGTCG AAATAAAAAC CAAGAGTAGC
  7861 CGGGCACGAT GGCACACTCG GGAGGCAGAG GCAGGCAGAT TTCTGAATTC AAGGCCAGCC
  7921 TGGTCTACAG AGTGAGTTCC AGGACAGCCA GGGCTACACA GAGAAACCCT GTCTCAGAAA
  7981 ACCAAAACAA ACAAACAAAC AAACAAAAAA CCCCAAGGGT ATAAAAGGAT TACCTGGTGT
  8041 TGGGTGACTG TAGTACCTGC TACTTAGAAT CCAGGAGTTT GGAATAGCCT TGGCAACAGA
  8101 GCAAACTCCA TCTCTTAAAA ATAAATAAAT AAATAAATAA AGGTAAAAAG AGAATGCAGG
  8161 GGTTAATCCT TCACACAGAA TCCCAGGGCC ACCCTGGGTG CCTGGCCCAG TGTGCAAACC
  8221 TCCAGAGGCC TCCAGGAAGG GCTGGAAGAG GACCCTGGGA GTAAGGAGGA CAATGTGATG
  8281 CCTGTGTCAC ACTTACTATC CTGTTGTGTC CCCATGCCCA TACATGGTCT TGCACAGGTC
  8341 CCCTCATAGC AGCCGTGCTC ACCCTGAGCA GAGCTCAGTC TCCACAGAGC CCAGAGTCAT
  8401 GCACCCTGCC TTCCTGCAGC AGAAGCCTGG AGAGGCATTT CCTGTTAGGG GGCAGCCTCC
  8461 TCCCGCTCTA AGCCCAGGTT CCCAGGGCCC CTGACTGAGC CAGGGTAAGG AGAGAGCAGG
  8521 CCTTGGCCTC CTCCCCCCTC CCTAACCCCT AATGCTGGTC CTGCTGGAGC CAAAAAAACT
  8581 TGGGAGTGAG GTAGGCTGAG CGAAGGGCTC ATCAAATACG TGTCTGGTTG AGGTGGTTCT
  8641 CAAGGAGGTT ATCAAGTTGA TTCTCACGCA GGGCCCAGGC AGGAATGGGT GGGGCCTCAT
  8701 CGTTGAGACC GTTTAGCAGG CCACATCATC CTAGCTTATC CACCTAAGGG CATCCCCAGC
  8761 ATGTCAGGAG AGCTGCGTGT GTGAGGCTGG GGTTTGGCGG CCTTAGATAG GGTGTCAGAG
  8821 AAGATGCTCT TGCCAGGCTT GGAAGGAGTC GTTCACATAT GTGTGATGAG AAAGGGCTGG
  8881 CCCGATCAAG TCGTTGAGCT CAGAATGCAG CCAGTATACT GATACTGGTG AATCCAGATC
  8941 CCGGCTTTCG GGAGTCTCTC TGGAAGACAG CTTCACCCCA GCTGCCTTAG CTGCAAACAA
  9001 CGCTGAAGTT TCAGAGGGTT GACTGGGGAT GCTTCTCAGA CAGACAGGTT CCCTTTTCCC
  9061 TGACTCTCAG CTCTGTAACG CCTGAGGTAG CCCGTTAGTT GTACCTCCCC CCTCCTCTGC
  9121 CCTTCGCCTC TCACCCCAGA ACCCCCCCCA CCCCACTGCT TCCTGCTCCA GCAGCCCCCG
  9181 GGGAGCTAGC AGGGGAGCTG GCAGCGGCCC CAGCCCACTC CTTACAAGGC GTGAGTCCGC
  9241 CGGGCCCGCC CCCGGCCCTC CCGCCAGAGG CCTTGATCCC TCCCCCTGTC AAGAGCAGCG

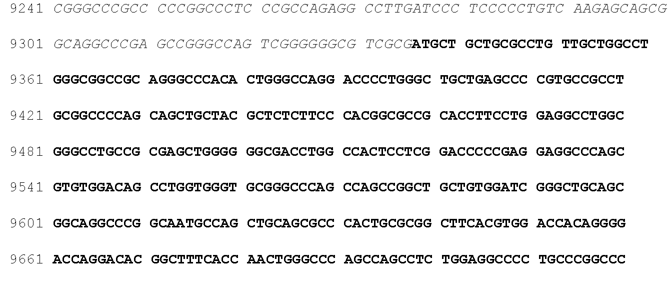

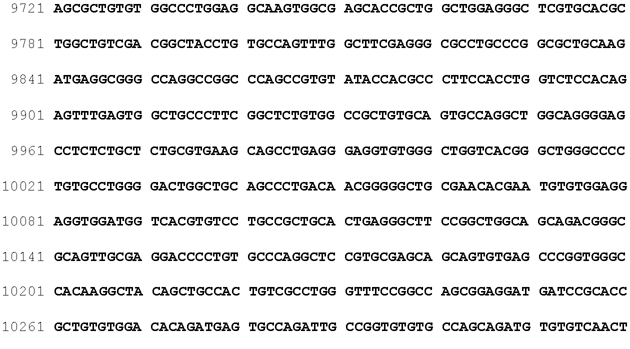

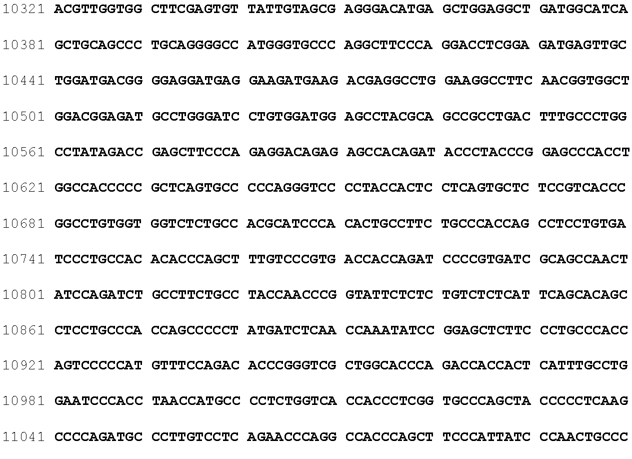

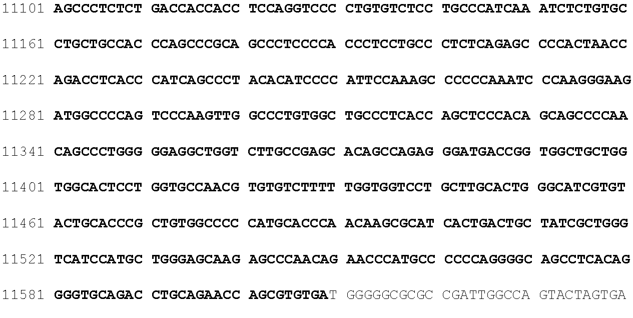

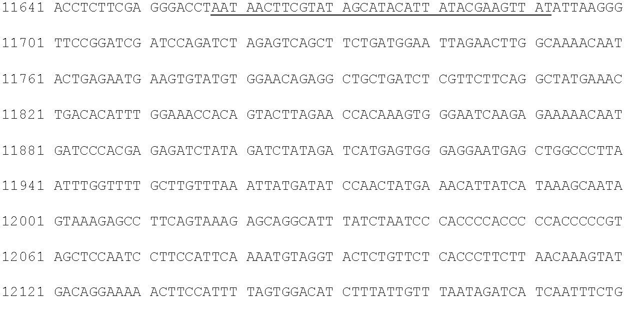





12181 CAGACTTACA GGACGGATCG ATCCCCTCAG AAGAACTCGT CAAGAAGGCG ATAGAAGGCG
12241 ATGCGCTGCG AATCGGGAGC GGCGATACCG TAAAGCACGA GGAAGCGGTC AGCCCATTCG
12301 CCGCCAAGCT CTTCAGCAAT ATCACGGGTA GCCAACGCTA TGTCCTGATA GCGGTCCGCC
12361 ACACCCAGCC GGCCACAGTC GATGAATCCA GAAAAGCGGC CATTTTCCAC CATGATATTC
12421 GGCAAGCAGG CATCGCCATG GGTCACGACG AGATCCTCGC CGTCGGGCAT GCGCGCCTTG
12481 AGCCTGGCGA ACAGTTCGGC TGGCGCGAGC CCCTGATGCT CTTCGTCCAG ATCATCCTGA
12541 TCGACAAGAC CGGCTTCCAT CCGAGTACGT GCTCGCTCGA TGCGATGTTT CGCTTGGTGG
12601 TCGAATGGGC AGGTAGCCGG ATCAAGCGTA TGCAGCCGCC GCATTGCATC AGCCATGATG
12661 GATACTTTCT CGGCAGGAGC AAGGTGAGAT GACAGGAGAT CCTGCCCCGG CACTTCGCCC
12721 AATAGCAGCC AGTCCCTTCC CGCTTCAGTG ACAACGTCGA GCACAGCTGC GCAAGGAACG
12781 CCCGTCGTGG CCAGCCACGA TAGCCGCGCT GCCTCGTCCT GCAGTTCATT CAGGGCACCG
12841 GACAGGTCGG TCTTGACAAA AAGAACCGGG CGCCCCTGCG CTGACAGCCG GAACACGGCG
12901 GCATCAGAGC AGCCGATTGT CTGTTGTGCC CAGTCATAGC CGAATAGCCT CTCCACCCAA
12961 GCGGCCGGAG AACCTGCGTG CAATCCATCT TGTTCAATGG CCGATCCCAT ATTGGCTGCA
13021 GGTCGAAAGG CCCGGAGATG AGGAAGAGGA GAACAGCGCG GCAGACGTGC GCTTTTGAAG
13081 CGTGCGAGAA TGCCGGGCCT CGGGAGGACC TTCGCGCCCG CCCCGCCCCT GAGCCCGCCC
13141 CTGAGCCCGC CCCCGGACCC ACCCCTTCCC AGCCTCTGAG CCCAGAAAGC GAAGGAGCAA
13201 AGCTGCTATT GGCCGCTGCC CCAAAGGCCT ACCCGCTTCC ATTGCTCAGC GGTGCTGTCC
13261 ATCTGCACGA GACTAGTGAG ACGTGCTACT TCCATTTGTC ACGTCCTGCA CGACGCGAGC
13321 TGCGGGGCGG GGGGGAACTT CCTGACTAGG GGAGGAGTAG AAGGTGGCGC GAAGGGGCCA
13381 CCAAAGAACG GAGCCGGTTG GCGCTACCGG TGGATGTGGA ATGTGTGCGA GGCCAGAGGC
13441 CACTTGTGTA GCGCCAAGTG CCAGCGGGGC TGCTAAAGCG CATGCTCCAG ACTGCCTTGG

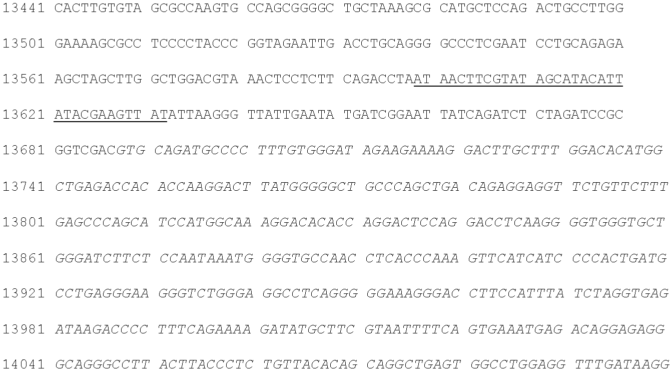

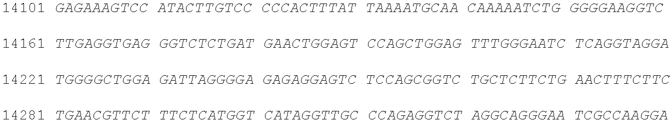


14341 TGCAGCCACC CTAGGGTTGT AGGTCTATCG CCTTCATAGA AGATAATAGG GGGAAGAGGT
14401 AAGAATAGGT TGAGGAAAGC CCAGTTCTAG CTTCCTGGGT CCACTTAGAA ACAAGGCCTT
14461 CCCACTAGGG AACACATTCT ACTCTTTACC CTGTTGGGGT GAAAGTGGCA CCGCTAGAAA
14521 AAAATCCCGG CAGACCCTAG CAGCACGAGG CTATTCAGGT TTGGGGTCCT GGTCAGAGAT
14581 CACGAGTGTG AGATAACCTC CCCCCCTTAT CCTCGCTGAC TGTCTTGGGG GGAAGGGACG
14641 GGACCTGGAA GAGGGAGACG CCGGTTCCAC CACCTCTATA ATTTTTCAGC TTAGCCCTCG
14701 TGGGGCTTCT AATTAGGACT GGGCAAAGCC GCACACCCCA CACCCCCCGC CCCCAGGCAG
14761 GTGAGGAGGG TGGCCCAGCG CCCTTTCTCT TCTTGAAGCC CCTATGCCAC ATTCTCGCCC
14821 AAGGATTGTA TCCCAGGAGA CTAGGAGAGG GAGTGCGGGC GCTGGGGACC AACTAGGAGT
14881 GGGGGTTCAC AGAGGGGGGG CAGAGGGAAC GCACCGCCCG GGTAGCCACT CTCACGTTCC
14941 CCTAACTCAA ACCAGACGCC CCTGCCAGAT CCTTCCCAGC TTTAACCTCG GAGAGGCAGT
15001 CGAGGGGGCG GGGGATTGAT GGATGGAATG GAGGGAGGTC CCCTGGTTTT CCAGAAAGCG
15061 TGGGCCTGAG GCACCACTTT TGGAATGACA CCGTCTGCTG GGACGGAGGC CTGAAGCTCC
15121 TTCCCATGAC CTCTGGCAGA GAGTAGTCAG CACCCTGAGG GAGAGAAAGA GTTAAATGCG
15181 CTGCAGTCCG CGGGAGGATG GGTGTGGGGA AGGGGGCCCA GAGCCGGCTC TTTGTCATCT
15241 AGTAATGAGC ACCAGATGCG GAGCTGCGTG CGGGCCTAAA CAAACGGCCT CCCAGGGCAG
15301 AGCCCCCGCC TGCACCCAGA CTCAGGCTCC GCCTCTACCG AAAGCTGAGT CTCAGGCTCC
15361 TCCCATTAAC TCTCAAGCTG AGCCTCACCT CCGCGCGGCG GAGGTTGCCT CGATTATCAG
15421 CCCCGCCCTT CCAATAATGA GCCACGCCCC TTGGAGGCGG GGGCGGGGTG GGGGAGAGAA
15481 AACTCCCCGG AGGCCCCGCC GGCCAAAGCC ATAGCCTCAC CCCTCGCCAG CTCAAAGATG
15541 CCCTTAGAGC TATTGCTAGC CCCTCTGCCT TATCACCTCC CCCAGGACCC CGCCCATAAG
15601 CAACAAGCTG CGTCTCCACA TCTAAGCAGA AATCGAGACA AGAGGAGTTT ATAAATCTTG

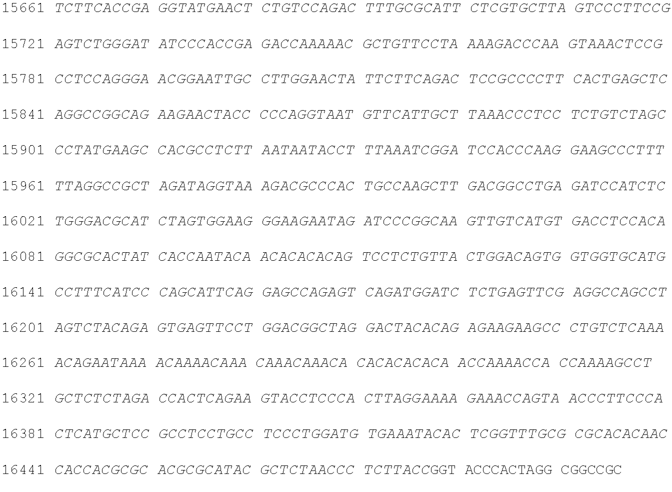

```

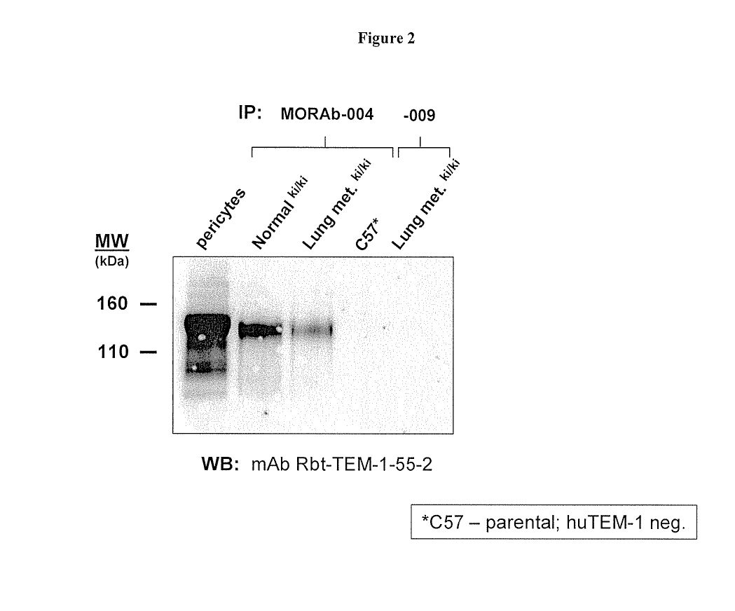

KpnI was used for linearizing the final vector prior to electroporation. The resultant construct was then linearized and introduced in mouse embryonic stem cell (ESC) line, and screened via polymerase chain reaction (PCR) for the integration of the construct into the mouse endosialin locus by using standard methods known to those skilled in the art of generating gene knock out or transgenic embryonic stem (ES) cells. Clones found to have the targeting construct integrated into the endogenous mouse endosialin gene locus were expanded and further validated by southern blot. Clone A was used to inject into mouse blastocysts and resultant mouse pups were screened using methods known to those skilled in the art, including genomic PCR or southern blotting using DNA extracted from tail clippings to identify those mice containing the targeting construct. These chimeric mice were then grown and crossed to C57BL/6 (also referred to as C57 mice) mice to generate heterozygotes and ultimately homozygous mice containing null murine endosialin and functional human endosialin expression. FIG. 2 shows the protein expression of human endosialin from HUE (lane 1-3) and wild type C57 (lane 4) mice. HUE mice were confirmed to have human endosialin expression patterns as those of the mouse endosialin gene as determined using wild type control C57 mice.

### Example 2

To demonstrate the utility of HUE analytical mice to screen for agents capable of binding human endosialin, HUE mice were employed in two different cancer models. One model (named SC model) entails the grafting of syngenic tumors implanted subcutaneously into the flank of HUE and wild type C57 control mice, the latter of which has intact mouse endosialin and does not express human endosialin. The other model (Lung Met model) employed the use of C57 syngenic metastatic Lewis lung carcinoma cells, which are stably transduced to express luciferase that can be used to measure tumor volume via in vivo bioluminescence imaging (Min, J, et. al. Nat. Med. 2010 March 16(3): 286-294). These cells, when introduced into HUE or C57 control mice, have been previously shown to be capable of metastasis to the lung of the host animal. See also Jenkins et al, Clin Exp Metastasis. 2003; 20(8):733-44 (human tumor cell lines engineered to express luciferase grafted onto SCID mouse and measured for bioluminescence using bioluminescent imaging (BLI) in response to treatment) which is incorporated herein by reference in its entirety.

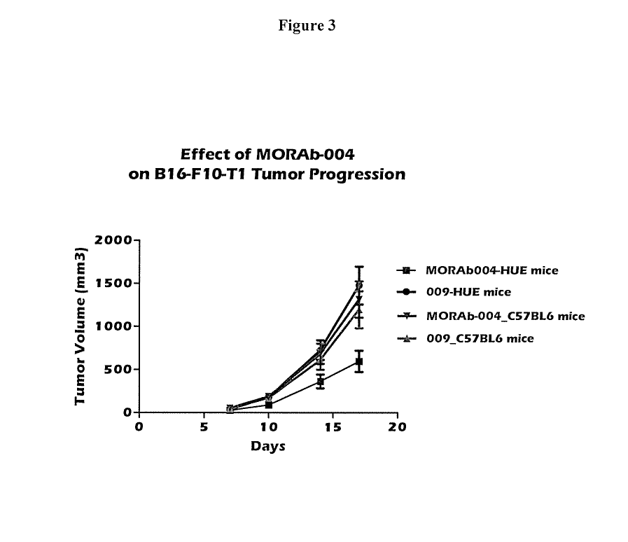

In the SC model, after successful implantation of tumor cells into the flank, mice were treated with the humanized anti-human endosialin (MORAb-004) antibody that does not recognize mouse endosialin or a control antibody and tested for the ability to suppress tumor growth as measured on day 17. As shown in FIG. 3, HUE mice treated with the anti-human endosialin antibody showed reduced tumor growth as compared to mice treated with the control human IgG. In the same experiment, anti-human endosialin antibody MORAb-004, or control human IgG, showed no growth reduction of tumors implanted in wild type C57 mice, demonstrating the specificity of MORAb-004 as its activity is restricted to human and not mouse endosialin.

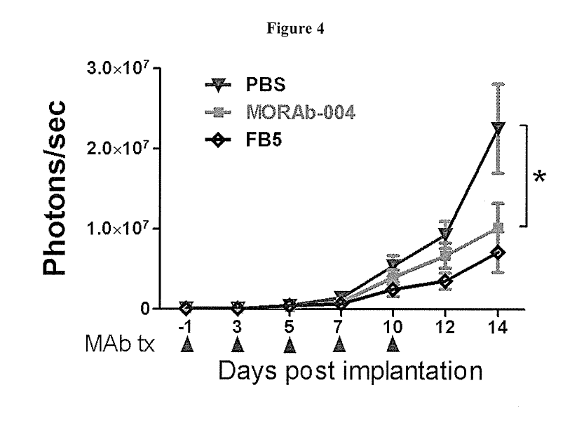

A second example of using HUE analytical mice is shown employing the Lung Met model. Cancer cells were introduced into HUE mice. The treatment with FB5, MORAb-004 or vehicle (phosphate buffer solution (PBS)) were initiated a day prior and continued on day 3, 5, 7, and 10. The magnitude of metastatic disease in the lung was quantified by in vivo bioluminescence imaging three times a week up to day 14. As shown in FIG. 4, mice treated with either anti-human endosialin antibody showed reduced metastatic disease as compared to the vehicle-treated mice.

In summary, the data described above demonstrate the utility of HUE mice when employed as analytical mice to identify agents that can target human endosialin for discovery, development and validation.

### Example 3

HUE mice generated in accordance with Example 1 were injected with B16F10T1 cells to the flanks subcutaneously. The animals received 5 doses at 40 mg/kg of MORAb-004 or a control of PBS only consecutively starting on day 3 post tumor cell implantations. To assess the tumor vasculature upon treatment, X-ray micro-CT was used. Micro-CT provides an overall analysis of tumor vasculature in the entire tumor and thus may overcome some limitations inherent in some other approaches such as immunohistochemistry.

Seventeen days post implantation; the tumor-bearing animals received a 50 μl intraperitoneal injection of heparin 10 min before being euthanized. The thoracic cavity was opened, a small nick was made in the left atrium of the heart using a 20 G needle, and a polyethylene cannula (inner diameter, 0.58 mm; outer diameter, 0.96 mm) was passed through the left ventricle and manually secured in place. 15-20 ml of PBS was manually perfused at a rate of 6 ml min-1 to remove blood. MICROFIL (Carver, commercially available lead chromate latex), was prepared as recommended by the manufacturer. Mice were then manually perfused with 10 ml of MICROFIL at a rate of 2 ml min-1. The infused latex mixture was allowed to polymerize at room temperature for sixty minutes then the animals were refrigerated at 4-8 degrees Celsius overnight before dissection of tissues of interest. Dissected tumors were immersed in 10% neutral buffered formalin.

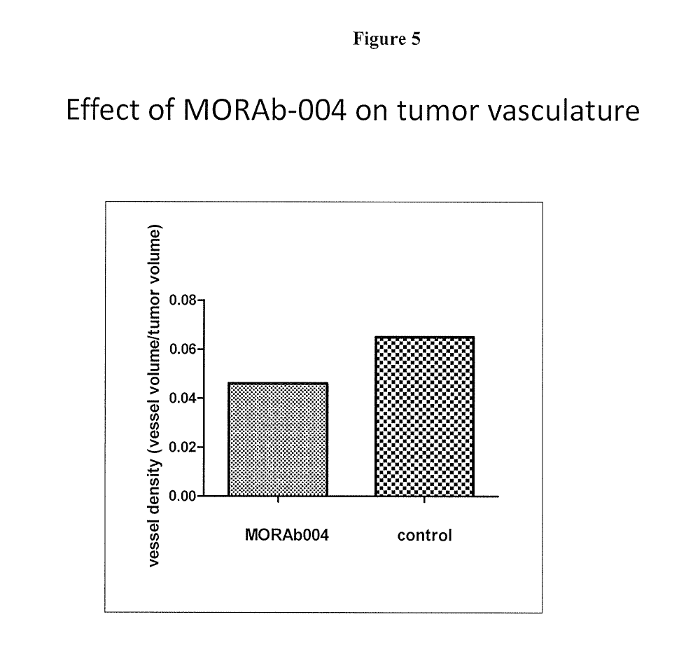

The tumors were then imaged with an X-ray micro-CT system performed by Numira Biosciences, and analyzed using image analysis software package (Altaview, Numira). The results can be seen in FIG. 5.

### REFERENCES

Asahara T, et. al. Endothelial progenitor cells for postnatal vasculogenesis. Am. J. Physiol. Cell Physiol. 2004; 287:C572-0579.
Bagley, et. al. Human endothelial precursor cells express tumor endothelial marker 1/endosialin/CD248, Mol Cancer Ther 2008; 7(8) 2536-2546.
Bagley, et. al. Endosialin/TEM 1/CD248 is a pericyte marker of embryonic and tumor neovascularization. Microvasc Res., 2008; 1-9.
Brady, et. al. Human endosialin (tumor endothelial marker 1) is abundantly expressed in highly malignant and invasive brain tumors. J Neuropatholgy and Exp Neurology, 2004; 63(12) 1274-1283.
Buhring H J, Seiffert M, Bock T A, Scheding S, Thiel A, Scheffold A, Kanz L, Brugger W. Expression of novel surface antigens on early hematopoietic cells. Ann N Y Acad. Sci. 1999 Apr. 30; 872:25-38; discussion 38-9.
Carson-Walter E B, Watkins D N, Nanda A, Vogelstein B, Kinzler K W, St Croix B. Cell surface tumor endothelial markers are conserved in mice and humans. Cancer Res. 2001 Sep. 15; 61(18):6649-55.
Christian S, Ahorn H, Koehler A, Eisenhaber F, Rodi H P, Garin-Chesa P, Park J E, Rettig W J, Lenter M C. Molecular cloning and characterization of endosialin, a C-type lectin-like cell surface receptor of tumor endothelium. J Biol. Chem. 2001 Mar. 9; 276(10):7408-14.
Conejo-Garcia J R, Buckanovich R J, Benencia F, Courreges M C, Rubin S C, Carroll R G, Coukos G. Vascular Leukocytes Contribute to Tumor Vascularization. Blood. 2005 Sep. 9; 105:679-1.
Das A, et. al. Retinal and choroidal angiogenesis: pathophysiology and strategies for inhibition. Prog. Retin. Eye Res., 2003; 22(6):721-748.
Dhanabal, et. al. Anti-angiogenic therapy as a cancer treatment paradigm. Curr. Med. Chem. Anti-Canc. Agents 2005; 5:115-130.
Florell S R, et. al. Preservation of RNA for functional genomic studies: a multidisciplinary tumor bank protocol. Mod. Pathol. 2001; 14(2):116-128.
Galiano R D, et. al. Topical vascular endothelial growth factor accelerates diabetic wound healing through increased angiogenesis and by mobilizing and recruiting bone marrow-derived cells. Am. J. Pathol. 2004; 164(6):1935-1947.
Kurosawa N, Kanemitsu Y, Matsui T, Shimada K, Ishihama H, Muramatsu T. Genomic analysis of a murine cell-surface sialomucin, MGC-24/CD164. Eur J. Biochem. 1999 Oct. 1; 265(1):466-72.
Kyriakos R J, et. al. The fate of antibodies bound to the surface of tumor cells in vitro. Cancer Res 1992; 52(4):835-842.
MacFadyen J R, et. al. Endosialin (TEM1, CD248) is a marker of stromal fibroblasts and is not selectively expressed on tumour endothelium. FEBS 2005; 579:2569-2575.
Marty, et al. Isolation and characterization of a scfv antibody specific for tumor endothelial marker 1 (tcm 1), a new reagent for targeted tumor therapy. Cancer Letters 2006; 235(2): 298-308.
Min J, Zaslaysky A, Fedele G, McLaughlin S K, Reczek E E, et. al. An oncogene-tumor suppressor cascade drives metastaticprostate cancer by coordinately activating Ras and NF-↓B. Nat. Med. 16: 286-294, 2010.
Opavsky R, Haviernik P, Jurkovicova D, Garin M T, Copeland N G, Gilbert D J, Jenkins N A, Bies J, Garfield S, Pastorekova S, Oue A, Wolff L. Molecular characterization of the mouse Tem1/endosialin gene regulated by cell density in vitro and expressed in normal tissues in vivo. J Biol. Chem. 2001 Oct. 19; 276(42):38795-807.
Paleolog, E. M., et al. Angiogenesis in arthritis: role in disease pathogenesis and as a potential therapeutic target. Angiogenesis 1999; 2:295-307.
Peters et al. Contribution of bone marrow-derived endothelial cells to human tumor vasculature. Nat. Med. 2005; 11:261-262.
Presta L G. Antibody engineering. Curr. Op. Struct. Biol. 1992; 2:593-596.
Queen C, et. al. A humanized antibody that binds to the interleukin 2 receptor. Proc. Nat. Acad. Sci. USA 1989; 86:10029-10033.
Rettig W J, Garin-Chesa P, Healey J H, Su S L, Jaffe E A, Old L J. Identification of endosialin, a cell surface glycoprotein of vascular endothelial cells in human cancer. Proc Natl Acad Sci U S A. 1992 Nov. 15; 89(22):10832-6.
St Croix B, Rago C, Velculescu V, Traverso G, Romans K E, Montgomery E, Lal A, Riggins G J, Lengauer C, Vogelstein B, Kinzler K W. Genes expressed in human tumor endothelium. Science. 2000 Aug. 18; 289(5482):1121-2.
Tomkowicz B, Rybinski K, Foley B, Ebel W, Kline B, Routhier E, Sass P, Nicolaides N C, Grasso L, and Zhou Y. Expression of Human Endosialin (CD248) Promotes Cellular Adhesion to Fibronectin Through Direct Binding and Activation of b₁ Integrins. Proc Natl Acad. Sci. 2007 104:17965-970.
Tomkowicz B, Rybinski K, Sebeck D, Sass P M, Nicolaides N C, Grasso L, and Zhou Y. Endosialin/TEM-1/CD248 Regulates Pericyte Proliferation through PDGF Receptor Signaling. Cancer Biol. Ther. 2010 9:1-8.
Watt S M, Chan J Y. CD164—a novel sialomucin on CD34+ cells. Leuk Lymphoma. 2000 March; 37(1-2):1-25.
Watt S M, Buhring H J, Rappold I, Chan J Y, Lee-Prudhoe J, Jones T, Zannettino A C, Simmons P J, Doyonnas R, Sheer D, Butler L H. CD164, a novel sialomucin on CD34(+) and erythroid subsets, is located on human chromosome 6q21. Blood. 1998 Aug. 1; 92(3):849-66.
Wilkinson-Berka J L, et. al. Vasoactive factors and diabetic retinopathy: vascular endothelial growth factor, cycloloxygenase-2 and nitric oxide. Curr. Pharm. Des. 2004; 10(27):3331-3348.
Yancopoulos G D, et. al. Vascular-specific growth factors and blood vessel formation. Nature 2000; 407(6801):242-248.
Zannettino A C, Buhring H J, Niutta S, Watt S M, Benton M A, Simmons P J. The sialomucin CD164 (MGC-24v) is an adhesive glycoprotein expressed by human hematopoietic progenitors and bone marrow stromal cells that serves as a potent negative regulator of hematopoiesis. Blood. 1998 Oct. 15; 92(8):2609-12.

### Sequences of Invention

```
SEQ ID NO: 1 mouse endosialin or TEM1 cDNA (ACCESSION NM_054042)
      1 gtcaagagca gcggcaggcc cgagccgggc cagtcggggg gcgtcgcgat gctgctgcgc
    61 ctgctgctgg cctgggtggc cgcggtgccc gcactgggcc aggtcccctg gacgccggag
  121 cctcgagccg cgtgcggccc cagcagctgc tacgcgctct ttccccggcg ccgcacattc
  181 ctggaagctt ggcgggcgtg ccgcgaattg gggggcaacc tggccacacc gcggacccca
  241 gaggaggccc agcgtgtgga cagcctggtg ggggtcgggc cggccaacgg gctgctatgg
  301 attgggttgc agcggcaggc taggcaatgc cagccgcagc gcccactgcg gggcttcata
  361 tggaccacgg gagaccagga caccgccttc accaactggg cccagccggc tacggaagga
  421 ccctgcccag cccagcgctg tgcagccctt gaggccagcg gagagcatcg ctggctcgaa
  481 ggctcgtgca cactggctgt cgatggctac ctctgccagt ttggttttga gggtgcctgc
  541 cctgccttgc cgcttgaggt gggtcaggcc ggtcccgctg tctacaccac acccttcaac
  601 ctggtttcca gcgagttcga atggctgccc tttggctccg tggcagctgt gcagtgccaa
  661 gctggcaggg gagcttctct gctgtgcgtg aaacagcctt caggtggcgt gggctggtcc
  721 cagactggcc cgctgtgccc agggactggc tgtggtcctg acaatggggg ttgcgaacat
  781 gagtgtgtgg aagaggtgga cggtgctgtg tcctgccgct gcagtgaagg cttccgtcta
  841 gcagcagatg ggcacagttg tgaagacccc tgtgcccagg ccccctgtga gcagcagtgt
  901 gaacctggag ggccacaagg ctatagctgc cactgtcgcc ttggcttccg gccagctgag
  961 gatgatccac accgctgcgt ggacacggat gagtgccaga ttgctggtgt gtgccagcag
1021 atgtgtgtca actatgttgg tggctttgag tgttactgca gcgagggtca cgagcttgag
1081 gcagatggta tcagctgtag ccctgcagga gccatgggtg cccaggcttc ccaggatctc
1141 agagatgagt tgctggatga tggagaagaa ggggaggatg aagaggagcc ctgggaggac
1201 tttgatggca cctggacaga ggaacagggg atcctatggc tggcacctac acatccacct
1261 gactttggcc tgccctatag gcccaacttc ccacaggatg gagagcctca gagattgcac
1321 ctggagccta cctggccacc cccacttagt gcccccaggg gcccctacca ctcctcagtg
1381 gtgtctgcca cacggcccat ggtgatctct gccactcgac ccacactacc ttctgcccac
1441 aagacctctg ttatttcagc tacacgccca cccctgagcc ctgtccaccc acctgccatg
1501 gcccctgcca cacctccagc tgtgttctct gagcaccaga tccccaaaat caaggccaat
1561 tatccagacc tgccttttgg ccacaagcct gggataacct cggccactca cccagcacgg
1621 tctcctccgt accagccccc cattatctca accaactatc cccaagtctt ccctccccac
1681 caggccccta tgtctccaga tacccacact atcacttatt tgcctccagt cccccctcac
1741 cttgatcctg gggataccac ttctaaagcc catcaacacc ctttgctccc agatgctcca
1801 ggtatcagaa cccaggcccc ccagctttct gtctcagctc tccagccccc tcttcctacc
1861 aactccaggt cttctgtcca tgaaactcct gtgcctgctg ccaaccagcc cccagccttc
1921 ccttcttctc ccctcccccc tcagaggccc actaaccaga cctcatctat cagccctaca
1981 cattcctatt ccagagcccc tctagtccca agggaaggag ttcccagtcc caaatcagtg
2041 ccacagctgc cctcggtgcc ctccacagca gctccaacag ccctggcaga gtcaggtctt
2101 gcaggccaaa gccaaaggga tgaccgctgg ctgctggtgg cactcctggt gccaacatgt
2161 gtcttcttgg tggtgctgct tgccctgggc attgtgtact gcactcgctg tggctcccac
2221 gcacccaaca agcggatcac ggactgctat cgctgggtca cacatgctgg gaacaagagc
2281 tcaacagaac ccatgccccc cagaggcagc cttacagggg tacagacctg tagaaccagt
2341 gtgtgatggg gtgcagatgc ccctttgtgg gatagaagaa aaggacttgc tttggacaca
2401 tggctgagac cacaccaagg acttatgggg gctgcccagc tgacagagga ggttctgttc
2461 tttgagccca gcatccatgg caaaggacac accaggactc caggacctca aggggtgggt
2521 gctgggatct tctccaataa atggggtgcc aacctcaccc aaaaaaaaaa aaaaaaaaaa
2581 aaaaaaaaaa aaaaaaaaaa aaaaaaaaaa aaaaaaaaaa aaaaaaaaaa aaaaaaaaaa
2641 aaaaaaaaaa aaaaaaaaaa
SEQ ID NO: 2 mouse endosialin or TEM1 protein (ACCESSION NM_054042)
MLLRLLLAWVAAVPALGQVPWTPEPRAACGPSSCYALFPRRRTF
LEAWRACRELGGNLATPRTPEEAQRVDSLVGVGPANGLLWIGLQRQARQCQPQRPLRG
FIWTTGDQDTAFTNWAQPATEGPCPAQRCAALEASGEHRWLEGSCTLAVDGYLCQFGF
EGACPALPLEVGQAGPAVYTTPFNLVSSEFEWLPFGSVAAVQCQAGRGASLLCVKQPS
GGVGWSQTGPLCPGTGCGPDNGGCEHECVEEVDGAVSCRCSEGFRLAADGHSCEDPCA
QAPCEQQCEPGGPQGYSCHCRLGFRPAEDDPHRCVDTDECQIAGVCQQMCVNYVGGFE
CYCSEGHELEADGISCSPAGAMGAQASQDLRDELLDDGEEGEDEEEPWEDFDGTWTEE
QGILWLAPTHPPDFGLPYRPNFPQDGEPQRLHLEPTWPPPLSAPRGPYHSSVVSATRP
MVISATRPTLPSAHKTSVISATRPPLSPVHPPAMAPATPPAVFSEHQIPKIKANYPDL
PFGHKPGITSATHPARSPPYQPPIISTNYPQVFPPHQAPMSPDTHTITYLPPVPPHLD
PGDTTSKAHQHPLLPDAPGIRTQAPQLSVSALQPPLPTNSRSSVHETPVPAANQPPAF
PSSPLPPQRPTNQTSSISPTHSYSRAPLVPREGVPSPKSVPQLPSVPSTAAPTALAES
GLAGQSQRDDRWLLVALLVPTCVFLVVLLALGIVYCTRCGSHAPNKRITDCYRWVTHA
GNKSSTEPMPPRGSLTGVQTCRTSV
SEQ ID NO: 3 human endosialin or TEM1 cDNA (ACCESSION NM_020404)
      1 agtccggggg catcgcgatg ctgctgcgcc tgttgctggc ctgggcggcc gcagggccca
    61 cactgggcca ggacccctgg gctgctgagc cccgtgccgc ctgcggcccc agcagctgct
  121 acgctctctt cccacggcgc cgcaccttcc tggaggcctg gcgggcctgc cgcgagctgg
  181 ggggcgacct ggccactcct cggacccccg aggaggccca gcgtgtggac agcctggtgg
  241 gtgcgggccc agccagccgg ctgctgtgga tcgggctgca gcggcaggcc cggcaatgcc
  301 agctgcagcg cccactgcgc ggcttcacgt ggaccacagg ggaccaggac acggctttca
  361 ccaactgggc ccagccagcc tctggaggcc cctgcccggc ccagcgctgt gtggccctgg
  421 aggcaagtgg cgagcaccgc tggctggagg gctcgtgcac gctggctgtc gacggctacc
  481 tgtgccagtt tggcttcgag ggcgcctgcc cggcgctgca agatgaggcg ggccaggccg
  541 gcccagccgt gtataccacg cccttccacc tggtctccac agagtttgag tggctgccct
  601 tcggctctgt ggccgctgtg cagtgccagg ctggcagggg agcctctctg ctctgcgtga
  661 agcagcctga gggaggtgtg ggctggtcac gggctgggcc cctgtgcctg gggactggct
  721 gcagccctga caacgggggc tgcgaacacg aatgtgtgga ggaggtggat ggtcacgtgt
  781 cctgccgctg cactgagggc ttccggctgg cagcagacgg gcgcagttgc gaggacccct
  841 gtgcccaggc tccgtgcgag cagcagtgtg agcccggtgg gccacaaggc tacagctgcc
  901 actgtcgcct gggtttccgg ccagcggagg atgatccgca ccgctgtgtg gacacagatg
  961 agtgccagat tgccggtgtg tgccagcaga tgtgtgtcaa ctacgttggt ggcttcgagt
1021 gttattgtag cgagggacat gagctggagg ctgatggcat cagctgcagc cctgcagggg
1081 ccatgggtgc ccaggcttcc caggacctcg gagatgagtt gctggatgac ggggaggatg
1141 aggaagatga agacgaggcc tggaaggcct tcaacggtgg ctggacggag atgcctggga
1201 tcctgtggat ggagcctacg cagccgcctg actttgccct ggcctataga ccgagcttcc
1261 cagaggacag agagccacag ataccctacc cggagcccac ctggccaccc ccgctcagtg
1321 cccccagggt cccctaccac tcctcagtgc tctccgtcac ccggcctgtg gtggtctctg
1381 ccacgcatcc cacactgcct tctgcccacc agcctcctgt gatccctgcc acacacccag
1441 ctttgtcccg tgaccaccag atccccgtga tcgcagccaa ctatccagat ctgccttctg
1501 cctaccaacc cggtattctc tctgtctctc attcagcaca gcctcctgcc caccagcccc
1561 ctatgatctc aaccaaatat ccggagctct tccctgccca ccagtccccc atgtttccag
1621 acacccgggt cgctggcacc cagaccacca ctcatttgcc tggaatccca cctaaccatg
1681 cccctctggt caccaccctc ggtgcccagc taccccctca agccccagat gcccttgtcc
1741 tcagaaccca ggccacccag cttcccatta tcccaactgc ccagccctct ctgaccacca
1801 cctccaggtc ccctgtgtct cctgcccatc aaatctctgt gcctgctgcc acccagcccg
1861 cagccctccc caccctcctg ccctctcaga gccccactaa ccagacctca cccatcagcc
1921 ctacacatcc ccattccaaa gccccccaaa tcccaaggga agatggcccc agtcccaagt
1981 tggccctgtg gctgccctca ccagctccca cagcagcccc aacagccctg ggggaggctg
2041 gtcttgccga gcacagccag agggatgacc ggtggctgct ggtggcactc ctggtgccaa
2101 cgtgtgtctt tttggtggtc ctgcttgcac tgggcatcgt gtactgcacc cgctgtggcc
2161 cccatgcacc caacaagcgc atcactgact gctatcgctg ggtcatccat gctgggagca
2221 agagcccaac agaacccatg ccccccaggg gcagcctcac aggggtgcag acctgcagaa
2281 ccagcgtgtg atggggtgca gacccccctc atggagtatg gggcgctgga cacatggccg
2341 gggctgcacc agggacccat gggggctgcc cagctggaca gatggcttcc tgctccccag
2401 gcccagccag ggtcctctct caaccactag acttggctct caggaactct gcttcctggc
2461 ccagcgctcg tgaccaagga tacaccaaag cccttaagac ctcagggggc gggtgctggg
2521 gtcttctcca ataaatgggg tgtcaacctt acccaaggaa aaaaaaaaaa aaaaaa
SEQ ID NO: 4 human endosialin or TEM1 protein (ACCESSION NM_020404)
MLLRLLLAWAAAGPTLGQDPWAAEPRAACGPSSCYALFPRRRTFLEAWRACRELGGDLATPRTPEEAQRVDSLVGAG
PASRLLWIGLQRQARQCQLQRPLRGFTWTTGDQDTAFTNWAQPASGGPCPAQRCVALEASGEHRWLEGSCTLAVDGY
LCQFGFEGACPALQDEAGQAGPAVYTTPFHLVSTEFEWLPFGSVAAVQCQAGRGASLLCVKQPEGGVGWSRAGPLCL
GTGCSPDNGGCEHECVEEVDGHVSCRCTEGFRLAADGRSCEDPCAQAPCEQQCEPGGPQGYSCHCRLGFRPAEDDPH
RCVDTDECQIAGVCQQMCVNYVGGFECYCSEGHELEADGISCSPAGAMGAQASQDLGDELLDDGEDEEDEDEAWKAF
NGGWTEMPGILWMEPTQPPDFALAYRPSFPEDREPQIPYPEPTWPPPLSAPRVPYHSSVLSVTRPVVVSATHPTLPS
AHQPPVIPATHPALSRDHQIPVIAANYPDLPSAYQPGILSVSHSAQPPAHQPPMISTKYPELFPAHQSPMFPDTRVA
GTQTTTHLPGIPPNHAPLVTTLGAQLPPQAPDALVLRTQATQLPIIPTAQPSLTTTSRSPVSPAHQISVPAATQPAA
LPTLLPSQSPTNQTSPISPTHPHSKAPQIPREDGPSPKLALWLPSPAPTAAPTALGEAGLAEHSQRDDRWLLVALLV
PTCVFLVVLLALGIVYCTRCGPHAPNKRITDCYRWVIHAGSKSPTEPMPPRGSLTGVQTCRTSV
```

1. A transgenic rodent comprising a nucleotide sequence encoding human endosialin integrated into the genome of said rodent.

2. The transgenic rodent of claim 1 wherein said nucleotide sequence comprises SEQ ID NO: 3.

3. The transgenic rodent of claim 1 wherein said human endosialin comprises the amino acid sequence of SEQ ID NO: 4.

4. The transgenic rodent of claim 1 wherein said rodent is a mouse.

5. The transgenic rodent of claim 1 wherein said nucleotide sequence is located within said rodent's endogenous endosialin gene locus.

6. The transgenic rodent of claim 1 wherein said rodent's endogenous endosialin gene is disrupted and therefore nonfunctional due to integration of said nucleotide sequence.

7. The transgenic rodent of claim 1 wherein said nucleotide sequence is under the control of said rodent's endogenous gene expression regulatory sequences.

8. The transgenic rodent of claim 1 further comprising a reporter gene or a selectable marker.

9. A cell isolated from the transgenic rodent of claim 1.

10. The cell of claim 9 wherein said cell is isolated from normal tissue, malignant tissue, inflamed tissue or diseased eye.

11. A method of screening test pharmacological agents to identify a targeting agent for human endosialin comprising:
administering a test pharmacological agent to a transgenic rodent comprising a nucleotide sequence encoding human endosialin integrated into the genome of said rodent;
measuring human endosialin activity in said rodent; and
comparing said human endosialin activity to a control,
wherein an increase or decrease in human endosialin activity relative to said control is indicative of a targeting agent for endosialin.

12. A method of screening test pharmacological agents to identify a targeting agent for human endosialin comprising:
contacting a test pharmacological agent to a cell of a transgenic rodent comprising a nucleotide sequence encoding human endosialin integrated into the genome of said rodent;
measuring human endosialin activity in said cell; and
comparing said human endosialin activity to a control,
wherein an increase or decrease in human endosialin activity relative to said control is indicative of a targeting agent for endosialin.

13. A method of validating an agent for human endosialin comprising:
administering said agent to a transgenic rodent comprising a nucleotide sequence encoding human endosialin integrated into the genome of said rodent;
measuring human endosialin activity in said rodent; and
comparing said human endosialin activity to a control,
wherein an increase or decrease in human endosialin activity relative to said control validates the agent for endosialin.

14. A method of validating an agent for human endosialin comprising:
contacting said agent to a cell of a transgenic rodent comprising a nucleotide sequence encoding human endosialin integrated into the genome of said rodent;
measuring human endosialin activity in said cell; and
comparing said human endosialin activity to a control,
wherein an increase or decrease in human endosialin activity relative to said control validates the agent for endosialin.

15. A method for screening for test agents that can suppress disease, wherein said disease is cancer, inflammatory disease, eye disease or reduced wound healing, said method comprising:
administering a test agent to a transgenic rodent comprising a nucleotide sequence encoding human endosialin integrated into the genome of said rodent wherein said rodent exhibits said disease;
measuring presence of said disease in said transgenic rodent; and
comparing presence of said disease in said transgenic rodent to a control,
wherein a decrease in said disease relative to said control is indicative of an agent that can suppress said disease.

16. A method for screening for test agents that can suppress disease, wherein said disease is cancer, inflammatory disease, eye disease or reduced wound healing, said method comprising:
contacting a test agent to a cell of a transgenic rodent comprising a nucleotide sequence encoding human endosialin integrated into the genome of said rodent wherein said rodent exhibits said disease;
measuring presence of said disease in said cell; and
comparing presence of said disease in said cell to a control,
wherein a decrease in said disease relative to said control is indicative of an agent that can suppress said disease.

17. A method for validating an agent that can suppress disease, wherein said disease is cancer, inflammatory disease, eye disease or reduced wound healing, said method comprising:
administering the agent to a transgenic rodent comprising a nucleotide sequence encoding human endosialin integrated into the genome of said rodent wherein said rodent exhibits said disease;
measuring presence of said disease in said transgenic rodent; and
comparing presence of said disease in said transgenic rodent to a control,
wherein a decrease in said disease relative to said control validates an agent that can suppress said disease.

18. A method for validating an agent that can suppress disease, wherein said disease is cancer, inflammatory disease, eye disease or reduced wound healing, said method comprising:
contacting the agent to a cell of a transgenic rodent comprising a nucleotide sequence encoding human endosialin integrated into the genome of said rodent wherein said rodent exhibits said disease;
measuring presence of said disease in said cell; and
comparing presence of said disease in said cell to a control,
wherein a decrease in said disease relative to said control validates an agent that can suppress said disease.

19.-28. (canceled)

29. The method of claim 15 wherein a tumor is grafted onto said transgenic rodent.

30. The method of claim 15 wherein the transgenic rodent has metastases.

31. The method of claim 17 wherein a tumor is grafted onto said transgenic rodent.

32. The method of claim 17 wherein the transgenic rodent has metastases.


SEQUENCE LISTING (17 sequences)

SEQ ID NO: 1 - DNA, 2660 bp, Mus musculus
gtcaagagcagcggcaggcccgagccgggccagtcggggggcgtcgcgatgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtgatggggtgcagatgcccctttgtgggatagaagaaaaggacttgctttggacacatggctgagaccacaccaaggacttatgggggctgcccagctgacagaggaggttctgttctttgagcccagcatccatggcaaaggacacaccaggactccaggacctcaaggggtgggtgctgggatcttctccaataaatggggtgccaacctcacccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 765 aa, Mus musculus
MLLRLLLAWVAAVPALGQVPWTPEPRAACGPSSCYALFPRRRTFLEAWRACRELGGNLATPRTPEEAQRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAVYTTPFNLVSSEFEWLPFGSVAAVQCQAGRGASLLCVKQPSGGVGWSQTGPLCPGTGCGPDNGGCEHECVEEVDGAVSCRCSEGFRLAADGHSCEDPCAQAPCEQQCEPGGPQGYSCHCRLGFRPAEDDPHRCVDTDECQIAGVCQQMCVNYVGGFECYCSEGHELEADGISCSPAGAMGAQASQDLRDELLDDGEEGEDEEEPWEDFDGTWTEEQGILWLAPTHPPDFGLPYRPNFPQDGEPQRLHLEPTWPPPLSAPRGPYHSSVVSATRPMVISATRPTLPSAHKTSVISATRPPLSPVHPPAMAPATPPAVFSEHQIPKIKANYPDLPFGHKPGITSATHPARSPPYQPPIISTNYPQVFPPHQAPMSPDTHTITYLPPVPPHLDPGDTTSKAHQHPLLPDAPGIRTQAPQLSVSALQPPLPTNSRSSVHETPVPAANQPPAFPSSPLPPQRPTNQTSSISPTHSYSRAPLVPREGVPSPKSVPQLPSVPSTAAPTALAESGLAGQSQRDDRWLLVALLVPTCVFLVVLLALGIVYCTRCGSHAPNKRITDCYRWVTHAGNKSSTEPMPPRGSLTGVQTCRTSV

SEQ ID NO: 3 - DNA, 2576 bp, Homo sapiens
agtccgggggcatcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatggggtgcagacccccctcatggagtatggggcgctggacacatggccggggctgcaccagggacccatgggggctgcccagctggacagatggcttcctgctccccaggcccagccagggtcctctctcaaccactagacttggctctcaggaactctgcttcctggcccagcgctcgtgaccaaggatacaccaaagcccttaagacctcagggggcgggtgctggggtcttctccaataaatggggtgtcaaccttacccaaggaaaaaaaaaaaaaaaaaa

SEQ ID NO: 4 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 5 - protein, 10 aa, Homo sapiens
GYTFTDYVIH

SEQ ID NO: 6 - protein, 17 aa, Homo sapiens
YINPYDDDTTYNQKFKG

SEQ ID NO: 7 - protein, 17 aa, Homo sapiens
ARRGNSYDGYFDYSMDY

SEQ ID NO: 8 - protein, 11 aa, Homo sapiens
RASQNVGTAVA

SEQ ID NO: 9 - protein, 7 aa, Homo sapiens
SASNRYT

SEQ ID NO: 10 - protein, 10 aa, Homo sapiens
QQYTNYPMYT

SEQ ID NO: 11 - DNA, 16497 bp, Homo sapiens
ctagtgagtcgtattacgtagcttggcgtaatcatggtcatagctgtttcctgtgtgaaattgttatccgctcacaattccacacaacatacgagccggaagcataaagtgtaaagcctggggtgcctaatgagtgagctaactcacattacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttacgcgccctgtagcggcgcattaagcgcggcgggtgtggtggttacgcgcagcgtgaccgctacacttgccagcgccctagcgcccgctcctttcgctttcttcccttcctttctcgccacgttcgctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggagaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtagttaacaaaaaaaagcccgccgaagcgggctttattaccaagcgaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtccgtaatacgactcacttaaggcgtagtacgggccccctcggtccgctctagaactacgatccagacatgataagatacattgatgagtttggacaaaccacaactagaatgcagtgaaaaaaatgctttatttgtgaaatttgtgatgctattgctttatttgtaaccattataagctgcaataaacaagttagatcctagtggatctgcattccaccactgctcccattcatcagttccataggttggaatctaaaatacacaaacaattaggaatcagtagtttaacacattatacacttaaaaattttatatttaccttagagctttaaatctctgtaggtagtttgtccaattatgtcacaccacagaagtaaggtttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcagggtccatggcgaggacctgcaggtcgcagccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactaggcgcccgggctagcacgcgtgcttcaagtctcagccctgccattaattaattaagtaagtaagtaatgctcactgcttcccttctgtttccccatctacaaaaagatatgcgtaccaccaagaacgttccacttctggggtgatccagtatcaacagggaagctagcacgtgtggaaagcaagcaggcaaagactccaaggagatcctagcctcccaagggcatcttcaggagccaggctcttcacttctgtgctcattcagtcattcaacaagtccccaccaacaacaactttttgtttgtctgtttattttgagacagggtttctctgtgtagccctggtttttctgaaacccactctgtagaccagggtggtctcaaactcaggggtctgcctgcttctgcctcccaagggctaggattaaaggcatgcaccaccatgttgagttcttttgtttgttttttgttttgtttttaaagatttatgtattttatgtatgtgagtacactgttgctgtcttcagacacaccagaagagggcatcagatcccattacagatggttgtgagccaccatgtggttgctgggaattgatctcaggacctctggaagagcagtcagtgctcttaacaactgagccatctctccagcccattgtgttttgttttattgtttttgttttgttttgttttgttttgttttgttttgttttgttttaatagagtctcatttatctttttacatcccagcctggcctgaaagtcactatgcatctaagggtcactctaactttctattcctctgtctccaactctgaagtgctgggatttcaggcacttcttactaattcttacttacagttcttttgtttcagttcttttggtttggttttttttttttttttttttttttttcgagacagggtttctctgtgtagccctggctgtcctggaactcactctgtagaccaggctgtcctcgaactcagcaatctgcctgcctttgcctcccaagtgctgggatttaaagacgtgtgccaccactgcccggctcttacttacagttcttactaagcaaaggctctaggcccagaggctccaaggcacagcagggctcactgtgagtctgctggagcagtctcccaagatgggagttctccactgggcacaggatacctggcaataactggagatatgtttttgttgtcctaactagaaggatgctattcacatatgggtcaaagccagggcacagatcaaaccactcataactatatgtcactgaatggcaataatgccagctaggattgagggattctgggtaggagtcaggtcataagggggttcaagtcaacaacaaagcatataccatggcagagggaactggtgagaggtgggaggtagggggacctcagagagaggagagtcatgtgtgggggctcagggactattctgcttcaactgagacccaagaaagtgaaaggcaaagggggagggcatttggatgttgcaggaaggagagaacatgggatgaaagctcagtgtctggtgggcctaaggcttgacacagacactgctagaacaaacagaaggaccaagggagaaagggaggggagggctggctgagcgttagagacagatcacatagcaggactttgcttgcctctgagtgagacatggccagagagggtcgtgaacagaggtggctagatctggccaggtctgctttttctttgtgtgtacgtgggcaagattattttattttatcagcgtttttgcctgcatatactgtatgcactacatgtgtgcccagtgtatggaaggggtcagaagaggacatcagagtccctggaactggagttccagatggttatgagcctccgtgtgggtaacaggagcctagccctggctctttgcaagagcagcaagtactcttgaccactgagacagctctccagccctcttgttttttaacggttctccccataagggagaaaaggaggaagaggagtggggggcagggtcctaactaggagatggagtgtgaccgcggtggacagaaaggcaaaggtggtgctgtgaagtggtcacactcttgatctagttttgaaaacgatgccaacaggatttgctgagatactggaccaggggtgtgggagagagagaaggatgagctcaagctttggctctgctccagctgagggatgatggcctctgctctcctgagaaggggcgtcgccctgcatgctttgagcataagacttatgctcaaaggcaagtgctgtataaacagcagttctcacagcctggctgtgtctgcagtgccgacctgggcactgtgaggaagttagtgaaagcacccgggtccccactggcatccagatttgctcaggagactaccaaaccttggtcctgtacaaaacatatatgcaaacagggagggctataacacaatagcgggctgaagagtccgcacacatgtatccccatgttgtggagggggacaatggaaacctggcaaggtcacctacacctataatctcagtactgctgcacagagacctacacaaaagggaagaggaggcaggaaaagagaagagaaagggagggaagggaagtggaaaggattgaaataatgaagaggagaaagaagcgtgtgcgtgctcgtgtgtgcgtgcatgcgtgagtgtgtatcctagcacttgggaagcagaggcatttggatctctgtgagctccagaccagccagaactacatagtgtaaactacatagtgtctttaaaaaggggtggggtgtatgggggtgggacatgaacgaggactcagagagcctcgaactcacacaggttagtagcagagtatccaggccaggacagtgttccaaagaacaaaccttctctttcactttctcatccatgggtcatgagatttttagtgctgggaattcaacccaggaccagacatcaccctaccacgaagccacatccccagctggctcagaatctgcggagaatataaaggatcgtgtgttaatgccagactcagaggtgatgggaagaccaggactttaaggtcatcctggaccacacagtgagctccagcttagcctgggcaacatgtaagactttgcctcaaaaaatcaaaaacggacagggcgtagtggtgcatgcctttaatcccagcactcgggaggcagaagcaggcggatttctgagttcgaagccatcctggtctacagagtgagttccaggacagccaaggctatacagagaaaccctgtctcggaaaaataaataaataaaaaacgggctggagtgatgcctaagcagcgaagagtacttcttgctcttgcagaggacccaagtttggttcccagcacccacacggtagtttaaaaatacccgtaactccagttccagggaatttgatgccctcttctgacttctgaaggtaacaggcacacacacggtacacacgcatacatgcaggaaaaacactcataaacagaaagcaaaaatgtttttaaaagtcgaaataaaaaccaagagtagccgggcacgatggcacactcgggaggcagaggcaggcagatttctgaattcaaggccagcctggtctacagagtgagttccaggacagccagggctacacagagaaaccctgtctcagaaaaccaaaacaaacaaacaaacaaacaaaaaaccccaagggtataaaaggattacctggtgttgggtgactgtagtacctgctacttagaatccaggagtttggaatagccttggcaacagagcaaactccatctcttaaaaataaataaataaataaataaaggtaaaaagagaatgcaggggttaatccttcacacagaatcccagggccaccctgggtgcctggcccagtgtgcaaacctccagaggcctccaggaagggctggaagaggaccctgggagtaaggaggacaatgtgatgcctgtgtcacacttactatcctgttgtgtccccatgcccatacatggtcttgcacaggtcccctcatagcagccgtgctcaccctgagcagagctcagtctccacagagcccagagtcatgcaccctgccttcctgcagcagaagcctggagaggcatttcctgttagggggcagcctcctcccgctctaagcccaggttcccagggcccctgactgagccagggtaaggagagagcaggccttggcctcctcccccctccctaacccctaatgctggtcctgctggagccaaaaaaacttgggagtgaggtaggctgagcgaagggctcatcaaatacgtgtctggttgaggtggttctcaaggaggttatcaagttgattctcacgcagggcccaggcaggaatgggtggggcctcatcgttgagaccgtttagcaggccacatcatcctagcttatccacctaagggcatacacagcatgtcaggagagctgcgtgtgtgaggctggggtttggcggccttagatagggtgtcagagaagatgctcttgccaggcttggaaggagtcgttcacatatgtgtgatgagaaagggctggcccgatcaagtcgttgagctcagaatgcagccagtatactgatactggtgaatccagatcccggctttcgggagtctctctggaagacagcttcaccccagctgccttagctgcaaacaacgctgaagtttcagagggttgactggggatgcttctcagacagacaggttcccttttccctgactctcagctctgtaacgcctgaggtagcccgttagttgtacctcccccctcctctgcccttcgcctctcaccccagaaccccccccaccccactgcttcctgctccagcagcccccggggagctagcaggggagctggcagcggccccagcccactccttacaaggcgtgagtccgccgggcccgcccccggccctcccgccagaggccttgatccctccccctgtcaagagcagcggcaggcccgagccgggccagtcggggggcgtcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatgggggcgcgccgattggccagtactagtgaacctcttcgagggacctaataacttcgtatagcatacattatacgaagttatattaagggttccggatcgatccagatctagagtcagcttctgatggaattagaacttggcaaaacaatactgagaatgaagtgtatgtggaacagaggctgctgatctcgttcttcaggctatgaaactgacacatttggaaaccacagtacttagaaccacaaagtgggaatcaagagaaaaacaatgatcccacgagagatctatagatctatagatcatgagtgggaggaatgagctggcccttaatttggttttgcttgtttaaattatgatatccaactatgaaacattatcataaagcaatagtaaagagccttcagtaaagagcaggcatttatctaatcccaccccacccccacccccgtagctccaatccttccattcaaaatgtaggtactctgttctcacccttcttaacaaagtatgacaggaaaaacttccattttagtggacatctttattgtttaatagatcatcaatttctgcagacttacaggacggatcgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatattggctgcaggtcgaaaggcccggagatgaggaagaggagaacagcgcggcagacgtgcgcttttgaagcgtgcgagaatgccgggcctcgggaggaccttcgcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgctaccggtggatgtggaatgtgtgcgaggccagaggccacttgtgtagcgccaagtgccagcggggctgctaaagcgcatgctccagactgccttgggaaaagcgcctcccctacccggtagaattgacctgcaggggccctcgaatcctgcagagaagctagcttggctggacgtaaactcctcttcagacctaataacttcgtatagcatacattatacgaagttatattaagggttattgaatatgatcggaattatcagatctctagatccgcggtcgacgtgcagatgcccctttgtgggatagaagaaaaggacttgctttggacacatggctgagaccacaccaaggacttatgggggctgcccagctgacagaggaggttctgttctttgagcccagcatccatggcaaaggacacaccaggactccaggacctcaaggggtgggtgctgggatcttctccaataaatggggtgccaacctcacccaaagttcatcatccccactgatgcctgagggaagggtctgggaggcctcagggggaaagggaccttccatttatctaggtgagataagacccctttcagaaaagatatgcttcgtaattttcagtgaaatgagacaggagagggcagggccttacttaccctctgttacacagcaggctgagtggcctggaggtttgataagggagaaagtccatacttgtcccccactttattaaaatgcaacaaaaatctgggggaaggtcttgaggtgagggtctctgatgaactggagtccagctggagtttgggaatctcaggtaggatggggctggagattaggggagagaggagtctccagcggtctgctcttctgaactttcttctgaacgttctttctcatggtcataggttgcccagaggtctaggcagggaatcgccaaggatgcagccaccctagggttgtaggtctatcgccttcatagaagataatagggggaagaggtaagaataggttgaggaaagcccagttctagcttcctgggtccacttagaaacaaggccttcccactagggaacacattctactctttaccctgttggggtgaaagtggcaccgctagaaaaaaatcccggcagaccctagcagcacgaggctattcaggtttggggtcctggtcagagatcacgagtgtgagataacctcccccccttatcctcgctgactgtcttggggggaagggacgggacctggaagagggagacgccggttccaccacctctataatttttcagcttagccctcgtggggcttctaattaggactgggcaaagccgcacaccccacaccccccgcccccaggcaggtgaggagggtggcccagcgccctttctcttcttgaagcccctatgccacattctcgcccaaggattgtatcccaggagactaggagagggagtgcgggcgctggggaccaactaggagtgggggttcacagagggggggcagagggaacgcaccgcccgggtagccactctcacgttcccctaactcaaaccagacgcccctgccagatccttcccagctttaacctcggagaggcagtcgagggggcgggggattgatggatggaatggagggaggtcccctggttttccagaaagcgtgggcctgaggcaccacttttggaatgacaccgtctgctgggacggaggcctgaagctccttcccatgacctctggcagagagtagtcagcaccctgagggagagaaagagttaaatgcgctgcagtccgcgggaggatgggtgtggggaagggggcccagagccggctctttgtcatctagtaatgagcaccagatgcggagctgcgtgcgggcctaaacaaacggcctcccagggcagagcccccgcctgcacccagactcaggctccgcctctaccgaaagctgagtctcaggctcctcccattaactctcaagctgagcctcacctccgcgcggcggaggttgcctcgattatcagccccgcccttccaataatgagccacgccccttggaggcgggggcggggtgggggagagaaaactccccggaggccccgccggccaaagccatagcctcacccctcgccagctcaaagatgcccttagagctattgctagcccctctgccttatcacctcccccaggaccccgcccataagcaacaagctgcgtctccacatctaagcagaaatcgagacaagaggagtttataaatcttgtcttcaccgaggtatgaactctgtccagactttgcgcattctcgtgcttagtcccttccgagtctgggatatcccaccgagaccaaaaacgctgttcctaaaagacccaagtaaactccgcctccagggaacggaattgccttggaactattcttcagactccgccccttcactgagctcaggccggcagaagaactacccccaggtaatgttcattgcttaaaccctcctctgtctagccctatgaagccacgcctcttaataatacctttaaatcggatccacccaaggaagccctttttaggccgctagataggtaaagacgcccactgccaagcttgacggcctgagatccatctctgggacgcatctagtggaagggaagaatagatcccggcaagttgtcatgtgacctccacaggcgcactatcaccaatacaacacacacagtcctctgttactggacagtggtggtgcatgcctttcatcccagcattcaggagccagagtcagatggatctctgagttcgaggccagcctagtctacagagtgagttcctggacggctaggactacacagagaagaagccctgtctcaaaacagaataaaacaaaacaaacaaacaaacacacacacacaaccaaaaccaccaaaagcctgctctctagaccactcagaagtacctcccacttaggaaaagaaaccagtaacccttcccactcatgctccgcctcctgcctccctggatgtgaaatacactcggtttgcgcgcacacaaccaccacgcgcacgcgcatacgctctaaccctcttaccggtacccactaggcggccgc

SEQ ID NO: 12 - DNA, 32 bp, Artificial Sequence
  Synthetic primer
acacacgcgtgcttcaagtctcagccctgcca

SEQ ID NO: 13 - DNA, 26 bp, Artificial Sequence
  Synthetic primer
acaacatcgcgacgccccccgactgg

SEQ ID NO: 14 - DNA, 32 bp, Artificial Sequence
  Synthetic primer
cacagtcgacgtgcagatgcccctttgtggga

SEQ ID NO: 15 - DNA, 32 bp, Artificial Sequence
  Synthetic primer
cacggatccggtaagagggttagagcgtatgc

SEQ ID NO: 16 - DNA, 32 bp, Artificial Sequence
  Synthetic primer
gggcgtcgcgatgctgctgcgcctgttgctgg

SEQ ID NO: 17 - DNA, 40 bp, Artificial Sequence
  Synthetic primer
caaaggcgcgcccccatcacacgctggttctgcaggtctg